SEQUENCE LISTING (11 sequences)

SEQ ID NO: 1 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 2 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 3 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 4 - protein, 609 aa, Homo sapiens [sequence omitted]

SEQ ID NO: 5 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 6 - protein, 347 aa, Homo sapiens
MSSWSRQRPKSPGGIQPHVSRTLFLLLLLAASAWGVTLSPKDCQVFRSDHGSSISCQPPAEIPGYLPADTVHLAVEFFNLTHLPANLLQGASKLQELHLSSNGLESLSPEFLRPVPQLRVLDLTRNALTGLPPGLFQASATLDTLVLKENQLEVLEVSWLHGLKALGHLDLSGNRLRKLPPGLLANFTLLRTLDLGENQLETLPPDLLRGPLQLERLHLEGNKLQVLGKDLLLPQPDLRYLFLNGNKLARVAAGAFQGLRQLDMLDLSNNSLASVPEGLWASLGQPNWDMRDGFDISGNPWICDQNLSDLYRWLQAQKDKMFSQNDTRCAGPEAVKGQTLLAVAKSQ

SEQ ID NO: 7 - protein, 105 aa, Homo sapiens
MVHLTPEEKSAVTALWGKVNVDAVGGEALGRLLVVYPWTQRFFESFGDLSTPDAVMGNPKVKAHGKKVLGAFSDGLAHLDNLKGTFATLSELHCDKLHVDPENFR

SEQ ID NO: 8 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 9 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 10 - protein, 1572 aa, Homo sapiens
MRTTKVYKLVIHKKGFGGSDDELVVNPKVFPHIKLGDIVEIAHPNDEYSPLLLQVKSLKEDLQKETISVDQTVTQVFRLRPYQDVYVNVVDPKDVTLDLVELTFKDQYIGRGDMWRLKKSLVSTCAYITQKVEFAGIRAQAGELWVKNEKVMCGYISEDTRVVFRSTSAMVYIFIQMSCEMWDFDIYGDLYFEKAVNGFLADLFTKWKEKNCSHEVTVVLFSRTFYDAKSVDEFPEINRASIRQDHKGRFYEDFYKVVVQNERREEWTSLLVTIKKLFIQYPVLVRLEQAEGFPQGDNSTSAQGNYLEAINLSFNVFDKHYINRNFDRTGQMSVVITPGVGVFEVDRLLMILTKQRMIDNGIGVDLVCMGEQPLHAVPLFKLHNRSAPRDSRLGDDYNIPHWINHSFYTSKSQLFCNSFTPRIKLAGKKPASEKAKNGRDTSLGSPKESENALPIQVDYDAYDAQVFRLPGPSRAQCLTTCRSVRERESHSRKSASSCDVSSSPSLPSRTLPTEEVRSQASDDSSLGKSANILMIPHPHLHQYEVSSSLGYTSTRDVLENMMEPPQRDSSAPGRFHVGSAESMLHVRPGGYTPQRALINPFAPSRMPMKLTSNRRRWMHTFPVGPSGEAIQIHHQTRQNMAELQGSGQRDPTHSSAELLELAYHEAAGRHSNSRQPGDGMSFLNFSGTEELSVGLLSNSGAGMNPRTQNKDSLEDSVSTSPDPMPGFCCTVGVDWKSLTTPACLPLTTDYFPDRQGLQNDYTEGCYDLLPEADIDRRDEDGVQMTAQQVFEEFICQRLMQGYQIIVQPKTQKPNPAVPPPLSSSPLYSRGLVSRNRPEEEDQYWLSMGRTFHKVTLKDKMITVTRYLPKYPYESAQIHYTYSLCPSHSDSEFVSCWVEFSHERLEEYKWNYLDQYICSAGSEDFSLIESLKFWRTRFLLLPACVTATKRITEGEAHCDIYGDRPRADEDEWQLLDGFVRFVEGLNRIRRRHRSDRMMRKGTAMKGLQMTGPISTHSLESTAPPVGKKGTSALSALLEMEASQKCLGEQQAAVHGGKSSAQSAESSSVAMTPTYMDSPRKVSVDQTATPMLDGTSLGICTGQSMDRGNSQTFGNSQNIGEQGYSSTNSSDSSSQQLVASSLTSSSTLTEILEAMKHPSTGVQLLSEQKGLSPYCFISAEVVHWLVNHVEGIQTQAMAIDIMQKMLEEQLITHASGEAWRTFIYGFYFYKIVTDKEPDRVAMQQPATTWHTAGVDDFASFQRKWFEVAFVAEELVHSEIPAFLLPWLPSRPASYASRHSSFSRSFGGRSQAAALLAATVPEQRTVTLDVDVNNRTDRLEWCSCYYHGNFSLNAAFEIKLHWMAVTAAVLFEMVQGWHRKATSCGFLLVPVLEGPFALPSYLYGDPLRAQLFIPLNISCLLKEGSEHLFDSFEPETYWDRMHLFQEAIAHRFGFVQDKYSASAFNFPAENKPQYIHVTGTVFLQLPYSKRKFSGQQRRRRNSTSSTNQNMFCEERVGYNWAYNTMLTKTWRSSATGDEKFADRLLKDFTDFCINRDNRLVTFWTSCLEKMHASAP

SEQ ID NO: 11 - protein, 175 aa, Homo sapiens
MLSHSSLTLAAPVLCAVLSSLPWRWRHLCCVPCYPTLLWRWRHLCCVPCYPLFPGTGGTCAVCRVTPLFPGAGGTCAMCRVILSSLALVAPVLCAVLSSLPWRWWHLCCVLCYPLFPGAGGTCAMCRVILSSLALAARTLCAGVFTSSLWGIRLETCFLPALKGCNSFVLTVPLN

(19) United States
(12) Patent Application Publication
(10) Pub. No.: US 2009/0104602 A1
(43) Pub. Date: Apr. 23, 2009

(54) Diagnosis of Tuberculosis

(76) Inventors: Delmiro Fernandez-Reyes, London (GB); Sanjeev Krishna, London (GB); Daniel Agranoff, London (GB); Gary Russell Coulton, London (GB)

Correspondence Address: NIXON & VANDERHYE, PC, 901 NORTH GLEBE ROAD, 11TH FLOOR, ARLINGTON, VA, 22203, US

(21) Appl. No.: 11/920,966

(22) PCT Filed: May 23, 2006
(86) PCT No.: PCT/GB2006/001888

§ 371 Date: Apr. 21, 2008

(30) Foreign Application Priority Data
May 23, 2005 (GB) 0510511.9

(51) Int. Cl. C12Q 1/68

(52) U.S. Cl. 435/6

(57) ABSTRACT

The invention provides a method of diagnosing tuberculosis (TB) in a test subject, said method comprising: (i) providing expression data of two or more markers in a subject, wherein at least two of said markers are selected from transthyretin, neopterin, C-reactive protein (CRP), serum amyloid A (SAA), serum albumin, apoliopoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp667I032; and (ii) comparing said expression data to expression data of said marker from a group of control subjects, wherein said control subjects comprise patients suffering from inflammatory conditions other than TB, thereby determining whether or not said test subject has TB.

### FIELD OF THE INVENTION

The present invention relates to the diagnosis of tuberculosis (TB).

### BACKGROUND OF THE INVENTION

Latent TB is present in one third of the world's population with a prevalence of active TB in many geographic areas exceeding 700 cases per 100,000 of the population (WHO Stop TB www.who.int/grb). This global TB epidemic is fuelled through synergy with HIV, which is found in 40%-70% of African patients with active TB. In areas of high TB prevalence, sputum smear microscopy is often the only available and affordable test but at best achieves a sensitivity of 50%. Culture of Mycobacterium tuberculosis, the diagnostic gold standard, increases sensitivity by a further 25%. Tuberculin skin tests are often insufficiently accurate to aid diagnosis, particularly in areas of high TB prevalence. Serological tests for TB have focused on detection of mycobacterial antigen(s) and, like skin tests, are frequently confounded by cross-reactivity with non-pathogenic mycobacteria or previous immunisation with BCG.

Most deaths from tuberculosis (TB) are preventable by early diagnosis and treatment. Early diagnosis also minimises morbidity and risk of transmission and commonly relies on microscopic identification of Mycobacterium tuberculosis. However microscopy is insensitive and culture of organisms is often too slow to aid therapeutic decisions. Recently developed DNA amplification and interferon-gamma based tests are expensive and need particular expertise.

An accurate and rapid diagnostic test for TB will have immense impact on the control of this disease.

### SUMMARY OF THE INVENTION

The present inventors have applied supervised machine-learning analysis to proteomic profiles, and have successfully distinguished patients with active TB from control patients with overlapping clinical features. The inventors have achieved a diagnostic accuracy of 94% for patients with TB and this is unaffected by ethnicity or HIV status. After ranking the most informative peaks in the proteomic profiles by feature selection, four polypeptides, serum amyloid A protein, transthyretin apolipoprotein-A1 and serum albumin, were identified and quantitated by immunoassay. Two of these polypeptides, serum amyloid A and transthyretin, reflect inflammatory states, and so the inventors also quantitated neopterin and C reactive protein. In addition, apolipoprotein-A2, hemoglobin beta, haptoglobin protein, DEP domain protein, leucine-rich alpha-2-glycoprotein and hypothetical protein DFKZp6671032 were identified as markers of TB by analysing the 2D gels used to identify peaks in the proteomic profile. Application of support vector machine classifiers to combinations of these markers gave a diagnostic accuracy of up to 84% for TB.

Accordingly, the present invention provides:

a method of diagnosing tuberculosis (TB) in a test subject, said method comprising:
(i) providing expression data of two or more markers in a test subject, wherein at least two of said markers are selected from transthyretin, neopterin, C-reactive protein (CRP), serum amyloid A (SAA), serum albumin, apoliopoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) determining whether expression of said markers is indicative of TB by comparing said expression data to expression data of said two or more markers from a group of control subjects, wherein said group of control subjects comprises patients suffering from inflammatory conditions other than TB,
   thereby determining whether or not said test subject has TB;

a method of a method of diagnosing tuberculosis (TB), said method comprising:
(i) providing expression data of two or more markers in a subject, wherein at least two of said markers are selected from transthyretin, neopterin, C-reactive protein (CRP), serum amyloid A (SAA), serum albumin, apolipoprotein-A1 (Apo-A1), apolipoprotein-A2 (Apo-A2), hemoglobin beta, haptoglobin protein, DEP domain protein, leucine-rich alpha-2-glycoprotein (A2GL (LRG1)) and hypothetical protein DFI<Zp6671032; and
(ii) determining whether expression of said markers is indicative of TB;

a method of diagnosing tuberculosis (TB), said method comprising:
(i) providing expression data of two or more markers in a subject, wherein at least two of said markers are selected from transthyretin, neopterin, C-reactive protein (CRP), serum amyloid A (SAA), serum albumin, apolipoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) determining whether expression of said markers is indicative of TB, wherein said determination is implemented using a computer system programmed with a trained machine learning classifier;

a computer-implemented method of diagnosing TB, said method comprising:
(i) inputting expression data of two or more markers in a subject; and
(ii) determining whether expression of said markers is indicative of TB using a computer system programmed with a trained support vector machine (SVM)
thereby diagnosing whether or not said patient has TB;

a method of training a support vector machine (SVM) classifier to diagnose tuberculosis (TB), said method comprising:
(i) providing training data which comprises:
   (a) training data relating to two or more markers in each of a first set of TB patients; and
   (b) training data relating to said two or more markers in each of a first set of control subjects;
(ii) using a SVM to discriminate the training data of TB patients from the training data of control subjects;
thereby training the SVM to diagnose TB;

an apparatus arranged to perform a method according to the invention comprising:
(i) means for receiving expression data of two or more markers in a sample from a subject;
(ii) a module for determining whether said data is indicative of TB, wherein said module comprises a trained machine learning classifier capable of distinguishing data from a TB patient from data from a control subject; and
(iii) means for indicating the results of said determination;

a computer program executable by a computer system, the computer program being capable, on execution by the computer system, of causing the computer system to perform a method according to the invention;

a storage medium storing in a form readable by a computer system having a computer program according to the invention;

a kit for diagnosing TB comprising:
(i) means for detecting two or more markers; and
(ii) a storage medium according to the invention;

a kit for diagnosing TB comprising:
(i) means for detecting two or more markers;
(ii) instructions for inputting data relating to detection of said markers into an apparatus according to the invention;

a kit for diagnosing TB comprising:
(i) means for detecting two or more markers selected from transthyretin, neopterin, C-reactive protein (CRP), serum amyloid A (SAA), serum albumin, apoliopoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032;

a method of identifying an agent for the treatment of TB, said method comprising:
(i) contacting a test agent with a TB marker selected from transthyretin, neopterin, CRP, SAA, serum albumin, Apo-A1, Apo-A2, hemoglobin beta, haptoglobin, DEP domain protein and A2GL; and
(ii) determining whether said test agent modulates the activity or expression of said marker,
   thereby determining whether or not said test agent is suitable for use in the treatment of TB; and

a method of identifying an agent for the treatment of TB, said method comprising:
(i) contacting cells ex vivo or in vivo with Mycobacterium tuberculosis and a test agent;
(i) monitoring expression of one or more TB markers selected from transthyretin, neopterin, CRP, SAA, serum albumin, Apo-A1, Apo-A2, hemoglobin beta, haptoglobin, DEP domain protein and A2GL; and
(ii) determining whether test agent modulates the expression of said one or more test markers,
   thereby determining whether or not said test agent is suitable for use in the treatment of TB.

### BRIEF DESCRIPTION OF THE FIGURES

FIG. 1 is a flow chart of a method of training a machine learning classifier.

FIG. 2 is a flow chart of a method of testing a trained machine learning classifier.

FIG. 3 is a flow chart of a method of determining whether a subject has or does not have TB using a trained machine learning classifier.

FIG. 4 shows the parameterisation of Gaussian kernel sigma value of Classifer (SVM_{—}1 in Table 3). The Gaussian SVM was trained with the initial training set (Table 2) using all mass peak clusters (10-fold cross validation for parameter selection). Classifier performance was then assessed on the initial testing set (Table 2).

FIG. 5 shows the averaged ROC using 10-fold train cross validation test. One hundred randomly selected train and test sets with a train:test ratio (80:20) were created. Parameters were selected using a 10-fold cross validation on the train set and performance obtained in the corresponding test set. a) Upper line shows the averaged ROC curve of the classifers obtained when kernel parameter is selected on sensitivity criteria. b) Upper line shows the averaged ROC curve of the classifiers obtained when kernel parameters is selected on specificity criteria.

### BRIEF DESCRIPTION OF THE SEQUENCES

SEQ ID NO: 1 is the amino acid sequence of human serum amyloid A1.

SEQ ID NO: 2 is the amino acid sequence of human C-reactive protein.

SEQ ID NO: 3 is the amino acid sequence of human transthyretin.

SEQ ID NO: 4 is the amino acid sequence of human serum albumin precursor.

SEQ ID NO: 5 is the amino acid sequence of human apolipoprotein-A1.

SEQ ID NO: 6 is the amino acid sequence of human leucine-rich alpha-2-glycoprotein.

SEQ ID NO: 7 is the amino acid sequence of human hemoglobin beta.

SEQ ID NO: 8 is the amino acid sequence of human haptoglobin.

SEQ ID NO: 9 is the amino acid sequence of human apolipoprotein-A2.

SEQ ID NO: 10 is the amino acid sequence of human DEP domain protein.

SEQ ID NO: 11 is the amino acid sequence of human hypothetical protein DFKZp6671032.

### DETAILED DESCRIPTION OF THE INVENTION

The present invention provides an ex vivo method of diagnosing tuberculosis (TB) in a test subject, said method comprising or consisting essentially of the steps of:
(i) providing expression data of two or more markers in a test subject, wherein at least two of said markers are selected from transthyretin, neopterin, C-reactive protein (CRP), serum amyloid A (SAA), serum albumin, apoliopoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) determining whether expression of said markers is indicative of TB by comparing said expression data to expression data of said marker from a group of control subjects, wherein said group of control subjects comprises patients suffering from inflammatory conditions other than TB,
   thereby determining whether or not said test subject has TB.

The group of control subjects may be selected from one or more patients with respiratory infections, patients with sarcoidosis, patients with inflammatory bowel disease, patients with malaria, patients with human African trypanosomiasis (HAT), patients with neurological disease, patients with autoimmune disease, patients with myeloma and healthy subjects.

The present invention provides an ex vivo method of diagnosing tuberculosis (TB), said method comprising or consisting essentially of the steps of:
(i) providing expression data of two or more markers in a subject, wherein at least two of said markers are selected from transthyretin, neopterin, C-reactive protein (CRP), serum amyloid A (SAA), serum albumin, apolipoprotein-A1 (Apo-A1), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) determining whether expression of said markers is indicative of TB, thereby diagnosing whether or not patient has TB.

A marker is a molecule, such as a protein or peptide, which is differentially expressed in a sample taken from a TB patient as compared to an equivalent sample or samples taken from one or more control subjects who do not have TB. The expression data typically provides an indication of the amount of marker present in a sample from a subject. A marker is present differentially in samples taken from TB patients and samples taken from control subjects if it is present at an increased level (positive marker) or a decreased level (negative marker) in TB samples compared to control samples. Preferably, the increase or decrease in the amount of a marker is a statistically significant difference.

The term ‘sensitivity’ is herein defined as the conditional probability of a true positive. The term ‘specificity’ is herein defined as the conditional probability of a true negative. The term ‘accuracy’ is herein defined as the proportion of correct classifications. Hence, accuracy indicates the reproducibility of the specific marker pairs or clusters for diagnosis of TB; sensitivity indicates how likely the combination was of achieving a true positive diagnosis; and specificity indicated how well each marker combination was in identifying samples as a true negative for TB infection.

Transthyretin, neopterin, CRP and SAA are known to be associated with pathophysiological processes in TB. However, it has not previously been suggested that any of these proteins may be used as markers in the diagnosis of TB. The present inventors have identified SAA, neopterin, CRP, serum albumin, Apo-A1, A2GL and DEP domain protein as positive markers of TB and transthyretin, Apo-A2, hemoglobin beta, haptoglobin and hypothetical protein DFKZp6671032 as negative markers of TB. The present inventors have found that when used in various combinations, these markers, and in particular SAA, neopterin, CRP and transthyretin, can be used to diagnose TB with a high degree of sensitivity, specificity and accuracy. Methods of the invention typically allow diagnosis of TB with an accuracy, a specificity and/or a sensitivity of at least 80%, for example, at least 85%, at least 90% or at least 95%.

The present invention thus allows determination of whether a subject is infected with Mycobacterium tuberculosis quickly and easily without the need to culture Mycobacterium tuberculosis in a sample from said subject. The method of the present invention enables TB to be distinguished from other infections such as viral and bacterial infectious and inflammatory diseases other than TB. Examples of infections and inflammatory diseases that may be distinguished from TB include other respiratory infections, sarcoidosis, inflammatory bowel disease, malaria, human African trypanosomiasis, neurological disease, autoimmune disease and myeloma.

In a method of the invention the expression data from the subject is typically compared to expression data of the same markers in a TB patient. The TB patient may have been diagnosed as having TB by culture of Mycobacterium tuberculosis from a sample from the patient. The expression data may also be compared to expression data of the same marker in one or more control subject. The control subject may be a patient having an inflammatory disease other than TB. The inflammatory disease may be caused by a pathogenic infection, for example a bacterial, viral or fungal infection. The control subject may have any of the diseases other than TB mentioned herein. Alternatively or additionally, one or more of the control subjects may be healthy individuals. A healthy individual is an individual not having an inflammatory disease.

Use of expression data from two or more markers enhances the accuracy of the diagnosis. Using combinations of more than two markers, such as three or more markers, may further enhance the accuracy of diagnosis. Accordingly, expression data from two or more markers, preferably three or more markers, for example four or more markers, such as five, six, seven, eight, nine, ten, fifteen, twenty or more markers, is used in a method of the invention. It is preferable that one of these markers used in the method of diagnosis is transthyretin. Preferred combinations include (i) transthyretin, SAA and CRP, (ii) transthyretin and neopterin and (iii) transthyretin, neopterin and CRP. Additional markers, such as serum albumin and/or Apo-A1, other than transthyretin, neopterin, SAA and CRP may be included in the analysis. Further additional markers include apolipoprotein-A2, hemoglobin beta, haptoglobin protein, DEP domain protein, A2GL and hypothetical protein DFKZp6671032.

Further additional markers may be proteins or peptides that are present at elevated or reduced levels in TB samples compared to control samples. The additional marker(s) may be characterised by an apparent molecular weight or mass-to-charge ratio (in/z value), for example as determined by mass spectrometry.

Such additional biomarkers may be identified by the method used by the present inventors to determine that SAA, serum albumin and Apo-A1 are positive markers of TB and that transthyretin is a negative marker of TB. Other positively and negatively correlated markers may be identified by surface enhanced laser desorption and ionization (SELDI) technology and supervised machine learning classification methods.

For example, the present inventors have identified ten positive markers and ten negative markers by comparing the proteomic signatures from TB patients with proteomic signatures from control subjects using a support vector machine classifier. The positive markers have m/z values of about M18394_{—}9, about M8952_{—}75, about M11720_{—}0, about M1144_{—}1, about M18591_{—}2, about M11488_{—}1, about M9076_{—}68, about M8895_{—}13, M10856_{—}8 and about M11541_{—}5 and the negatively correlated markers have m/z values of about M4100_{—}03, about M3898_{—}52, about M13972_{—}1, about M3322_{—}01, about M2956_{—}45, about M5644_{—}96, about M3939_{—}63, about M4056_{—}39, about M6649_{—}74 and about M13774_{—}3. The marker having an in/z value of about M11541_{—}5 is SAA. The marker having an m/z value of about M18394_{—}9 is serum albumin. The marker having an m/z value of about M11454_{—}1 is Apo-A1. The marker having an m/z value of about M13774_{—}3 is transthyretin. There may be some variation in m/z value. For example, there may be variation that is dependent on the resolution of the machine used to determine m/z value or on post-translational modification of the marker. Accordingly, the markers listed above may have the specified in/z value plus or minus about 10%, about 5%, about 1%, about 0.5% or about 0.2%.

The identity of the additional markers identified by SELDI analysis may be determined by tryptic digestion and Matrix-assisted laser desorption/ionization time of flight (MALDI-ToF) mass spectroscopy of the peptide mass fingerprints and comparison with protein databases such the MASCOT database. SAA1 has an m/z value of M11541_{—}5 and transthyretin has an m/z value of M13774_{—}3 and were identified by such methods.

The markers may also be identified by identifying the protein spots corresponding to the m/z value on a 2-dimensional (2D) gel and excising and identifying the protein present in the spot. The 2D gel may be obtained from pooled sera from a number, such as about 10, about 20 or more, of TB patients or a number, such as about 10, about 20 or more, of control subjects. The m/z value is generally slightly smaller than the passive elution (PE) mass. The increase in the PE mass over the m/z value is proportional to the time used to do the passive elution. Therefore, if this method is used it is important to note that the link between the m/z value and the PE mass is approximate. However, the identity of the marker may be confirmed by immunodepleting the original sample and repeating the SELDI-ToF analysis. A reduction in the size of the peak with the m/z value of interest indicates that a correct identification has been made. However, further identification is not essential for the proteins to be mass used as markers in a method of the invention. The positive markers having m/z values of M18394_{—}9 and M11454_{—}1 have been identified as serum albumin precursor and apolipoprotein A1 (Apo-A1) using this method. Thus one or more of the markers identified by their in/z values, including serum albumin and/or Apo1-A1, may be used as markers in a method of the invention.

Additional markers of TB may have been identified by identifying polypeptides that are differentially present in 2D gels containing serum proteins from TB patients and control subjects. The markers identified in this way are apolipoprotein A2 (Apo-A2), hemoglobin beta, haptoglobin protein, DEP domain protein and hypothetical protein (DFKZp6671032) and leucine-rich-alpha-2-glycoprotein (A2GL (LRG1)).

Following supervised machine learning analysis of proteomic signatures from TB patients and control subjects, the protein clusters suitable for use as markers of TB may be identified by any method which enables selection of protein clusters with the power to discriminate between TB patients and control subjects. Typically, a correlation filter method is used to detect independently informative peaks. For example, the Pearson correlation coefficient may be used to rank peaks for their discriminatory power. The Pearson correlation coefficient is defined as

$$R ( k ) = \frac{covariance ( {X}_{k} , Y )}{\sqrt{variance ( {X}_{k} ) variance ( Y )}}$$

where Xₖ is the random variable corresponding to the k^{th} component of sample input vectors x and Y is the random variable of output labels.

The estimate of R(k) is given by

$$\hat{R} ( k ) = \frac{{\sum }_{i = 1}^{m} ( {x}_{i , k} - {\overline{x}}_{k} ) ( {y}_{i} - \overline{y} )}{\sqrt{{\sum }_{i = 1}^{m} {( {x}_{i , k} - {\overline{x}}_{k} )}^{2} {\sum }_{i = 1}^{m} {( {y}_{i} - \overline{y} )}^{2}}}$$

where x_{i,k} correspond to value m/z of the mass cluster k of sample i, yᵢ is the class label for sample i and m is the number of samples. R(i) may be used a test statistic to assess the significance of a variable and it is linked to the t-test. \hat{R}(k) may be calculated between values of each mass cluster and corresponding class labels across the training set. \hat{R}(k) may then be used to rank positively and negatively correlated mass clusters. Mass clusters with the highest positive and/or highest negative correlation coefficients may be selected.

Proteins are often present in biological material in a plurality of different forms characterised by detectably different molecular masses. Hence, analysis of expressed proteins in a biological sample by methods such as SELDI detects the various different forms of the protein as a protein cluster. The different forms may result from pre-translational and/or post-translational modifications. For example, the transthyretin marker may be transthyretin precursor or mature transthyretin. As additional Examples, each of the serum albumin, Apo-A1 and Apo-A2 markers may also be a precursor or mature form of the protein, preferably a precursor form. Allelic variation, the generation of splice variants and RNA editing give rise to pre-translational modifications. Post-translational modifications include proteolytic cleavage, glycosylation, phosphorylation, lipidation, oxidation, methylation, cystinylation, sulphonation and acetylation. The expression data may relate to any one or more form of the protein. Pre- and/or post-translational modifications may give rise to fluctuations in the m/z value of a marker in SELDI-ToF.

In one embodiment of the invention, the expression data may relate to one or more peptide derived from the said markers. For example, the expression data of SAA may relate to expression of a peptide resulting from loss of the N-terminal arginine of SAA. The full sequence of SAA1 is shown in SEQ ID NO: 1.

The expression data may, in one embodiment, relate to a particular form of the marker. For example, the positive markers Apo-A1 may be the form having a molecular mass of about 11400 to about 11600 and/or the positive marker serum albumin may be the form having a molecular weight of about 18300 to about 18500 daltons (Da).

Expression data may be obtained by any suitable method. In one embodiment, the expression data indicates the presence or absence of each marker of interest. The expression data preferably provides an indication of the amount of each marker present in a sample from a subject, i.e. the data is quantitative. The expression data may additionally qualify the form of each marker, for example the form of the protein present.

Typically, expression data is obtained by capture of the markers on a solid phase, or surface, and detection of the captured markers. The surface is designed to select marker proteins from samples according to a general property of the markers being used or according to specific properties of the different protein markers. The surface is typically a bead, plate, membrane or chip on which one or more capture reagent is bound. The capture reagent may be a specific chromatographic surface. The chromatographic surface may be chemically or biochemically treated. Chemically treated surfaces may be anionic, cationic, hydrophobic, hydrophilic or metal. Such chemically treated surfaces are capable of capturing proteins with a particular chemical property. Such chemically treated surfaces may comprise, for example, ion exchange materials, metal chelators, such as nitriloacetic acid or iminodiacetic acid, immobilised metal chelates, hydrophobic interaction adsorbents, hydrophilic interaction adsorbents, dyes, simple biomolecules, such as nucleotides, amino acids, simple sugars and fatty acids, and mixed mode adsorbents, such as hydrophobic attraction/electrostatic repulsion adsorbents.

In an embodiment where the surface is biochemically treated, the capture reagent is typically a specific binding reagent for a particular marker. In this embodiment, the surface typically comprises a specific binding reagent for each marker being used. A protein “specifically binds” to a marker when it binds with preferential or high affinity to the marker for which it is specific but does not bind, does not substantially bind or binds with only low affinity to other substances. The specific binding capability of a protein may be determined by any suitable method. A variety of protocols for competitive binding are well known in the art (see, for example, Maddox et al. (1993)).

The specific binding agent may be an antibody or antibody fragment specific for the marker. Suitable antibodies are available in the art. Antibodies and antibody fragments may also be generated using standard procedures known in the art.

The antibody may be a monoclonal or polyclonal antibody. Monoclonal antibodies are preferred. The binding proteins may also be, or comprise, an affinity ligand or an antibody fragment, which fragment is capable of binding to the marker. Such antibody fragments include Fv, F(ab′) and F(ab′)₂ fragments as well as single chain antibodies. Aptamers, antibodies and interacting fusion proteins may also be used as specific binding agents. The specific binding agent may recognize one or more form of the marker of interest.

Other biochemically treated surfaces may be coated with a nucleic acid molecule, such as a polypeptide, a polysaccharide, a lipid, a steroid or a conjugate molecule, such as a glycoprotein, a lipoprotein, a glycolipid or a nucleic acid (e.g. DNA)-protein conjugate.

Methods for coupling specific binding agents such as antibodies to a surface are well known in the art.

The surface may be a protein chip array. A protein chip array comprises discrete spots, typically of a diameter of 2 mm, of capture reagents. The capture reagents at each spot on the array may be the same or different. Protein chip arrays suitable for use in the invention are well known in the art. For example, suitable chips are available from Ciphergen Biosystems and include CM10, IMAC-3, CM16, SAX2, H4, NP20, H50, Q-10, WCX-2, IMAC-30, LSAX-30, LWCX-30, IMAC-40, PS10, PS-20 and PG-20 protein chip arrays.

These protein biochips typically comprise an aluminium substrate in the form of a strip. The surface of the strip is coated with silicon dioxide. In the case of the NP-20 biochip, silicon oxide functions as a hydrophilic adsorbent to capture hydrophilic proteins. H4, H50, SAX-2, Q-10, WCX-2, CM-10, IMAC-3, IMAC-30, PS-10 and PS-20 biochips further comprise a functionalised, cross-linked polymer in the form of a hydrogel physically attached to the surface of the biochip or covalently attached through a silane to the surface of the biochip. The H4 biochip has isopropyl functionalities for hydrophilic binding. The H50 biochip has nonylphenoxylpoly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu²⁺ and Ni²⁺, by chelation. These immobilised metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalised latex beads on their surfaces.

The surface may be a well of a microtitre plate, such as a 96-well microtitre plate. Typically, each well of such a plate will comprise a different capture reagent, such as a different antibody, as each well may comprise two or more discrete spots of different antibodies.

The capture surface may be a column loaded with a plurality of beads coated with the capture reagent. Multiple columns, each able to capture a single marker protein may be used. Alternatively, a single column may contain beads coated with specific binding agents for different marker proteins, so that all marker proteins are captured in the same column.

A sample from a subject is typically brought into contact with the surface under conditions suitable for binding of marker proteins in the sample to the surface. The proteins present in the sample may optionally be fractionated and the fraction(s) comprising the markers being detected may be collected and brought into contact with the surface. Unbound material is washed away using an appropriate solvent or buffer, such as phosphate buffered saline (PBS), designed to elute unbound proteins and other substances whilst retaining the markers of interest bound to the surface. The sample from the subject is typically a blood, plasma or serum sample.

The captured marker proteins may be detected by any suitable method. In one embodiment, bound markers may be detected by an immunoassay, for example by an ELISA assay or fluorescence-based immunoassay. In a typical immunoassay, the bound marker may be detected using an antibody, or fragment thereof, which will bind to the marker. Where the capture reagent is an antibody, the detector antibody is typically a different antibody to the capture reagent. Typically, the antibody binds the marker at a site which is different to the site which binds the capture reagent. The antibody may be specific for the complex formed between the marker and the capture reagent immobilised on the support.

Generally, the antibody is labelled with a label that may be detected either directly or indirectly. A directly detectable label may comprise a fluorescent label such as fluoroscein, Texas red, rhodamine or Oregon green. The binding of a fluorescently labelled antibody to the immobilised capture reagent/marker complex may be detected by microscopy. For example, using a fluorescent, bifocal or confocal microscope.

Preferably, the antibody is conjugated to a label that may be detected indirectly. The label that may be detected indirectly may comprise an enzyme which acts on a precipitating non-fluorescent substrate that can be detected using an automated reader. An automated reader is typically based on a video camera and image analysis software. The automated reader is capable of providing a measure of the quantity of each detected marker. Preferred enzymes include alkaline phosphatase and horseradish peroxidase. Automated readers are well known in the art and include, for example the Grifols Tritorus analyser (Grifols, Cambridge UK).

Other indirect methods may be used to enhance the signal from the detector antibody. For example, the detector antibody may be biotinylated allowing detection using streptavidin conjugated to an enzyme such as alkaline phosphatase or horseradish peroxidase or streptavidin conjugated to a fluorescent probe such as FITC or Texas red.

In all detection steps, it is desirable to include an agent to minimise non-specific binding of the second and subsequent agent. For example bovine serum albumin (BSA) or foetal calf serum (FCS) may be used to block non-specific binding.

In one embodiment, the captured proteins may be detected by gas phase ion spectrometry, such as mass spectrometry, for example MALDI or SELDI, following elution of the proteins from the surface, e.g. chip or beads. Such detection methods enable different proteins and different forms of the same protein to be distinguished without the need for labelling.

Gas phase ion spectrometry requires a gas phase ion spectrometer to detect gas phase ions. Gas phase ion spectrometers include an ion source that supplies gas phase ions and include mass spectrometers, ion mobility spectrometers and total ion current measuring devices. A mass spectrometer is a gas phase ion spectrometer that measures a parameter which can be translated into mass-to-charge rations of gas phase ions. Mass spectrometers typically include an ion source and a mass analyser. Examples of mass spectrometers are time-of-flight (ToF), magnetic sector, quadrupole filter, ion trap, ion cyclotron resonance, electrostatic sector analyser and hybrids of these. A laser desorption mass spectrometer is a mass spectrometer which uses laser as a means to desorb, volatilize and ionize an analyte. A tandem mass spectrometer is mass spectrometer that is capable of performing two successive stages of in/z-based discrimination or measurement of ions, including ions in an ion mixture.

The captured markers may be desorbed or ionized from the capture surface using any suitable source of ionizing energy, such as high energy particles generated via beta decay of radionuclides or primary ions generating secondary ions. The preferred form of ionizing energy for solid phase analytes is a laser.

A preferred mass spectrometric technique for use in the invention is SELDI (Surface Enhanced Laser Desorption and Ionization) which is a method of desorption/ionization gas phase ion spectrometry in which the marker proteins are captured on the surface of a protein chip, or SELDI probe, that engages the probe interface of the gas phase ion spectrometer. In this embodiment using a protein chip array to capture the marker proteins, a protein chip reader may be used to detect the bound markers. Proteins bound on the protein chip are typically allowed to dry prior to the addition of an energy absorbing molecule (EAM) solution and the insertion of the protein chip into a protein chip reader to measure the molecular weights of the bound proteins. Upon laser activation in the protein chip reader, the sample becomes irradiated and the adsorption/ionization proceeds to liberate gaseous ions from the protein chip arrays. These gaseous ions enter the time of flight mass spectrometry (ToF MS) region of the protein chip reader which measures the mass-to-charge ratio (m/z) of each protein, based on its velocity through an ion chamber. Time lag focussing may be used to increase the mass accuracy of the signal output. Signal processing is accomplished by high speed analogue to digital converter, which is linked to a personal computer. Detected proteins are displayed as a series of peaks. The amplitude of the peaks is an indication of the amount of each protein present in a sample. Suitable EAMs for use in methods of the invention include cinnamic acid derivatives, sinapinic acid and dihydroxybenzoic acid.

Expression data may also be obtained by nephelemetry. Nephelemetry is a laboratory technique used to obtain a measurement of the amount of a marker accurately and rapidly. The data may, for example, be obtained by particle-enhanced immunonephelemetry or rate nephelemetry. The BNII analyser (Dade Behring, Milton Keynes, UK) is suitable for performing particle enhanced immunonephelemetry. The Beckman Image (Beckman Coniter, High Wycombe, UK) may be used to perform rate nephelemetry. The Beckman Image may be calibrated against the International Reference Preparation CRM 470. Measurement of marker expression may be carried out by following the instructions provided by the manufacturer of the analyser used.

Other detection methods that may be used include optical techniques, such as confocal or fluorescence microscopy, electrochemical techniques, such as voltametry and amperometry, atomic force microscopy and radio frequency techniques, such as multipolar resonance spectroscopy.

The expression pattern of the markers of interest is examined to determine whether expression of the markers is indicative of the patient having TB. Any suitable method of analysis may be used. Typically, the analysis method used comprises comparing the expression data obtained from a subject to expression data obtained from patients known to have TB and control subjects who do not have a Mycobacterium tuberculosis infection. It can then be determined whether or not the expression of the markers in the subject is more similar to the expression pattern observed in known TB patients or to the expression pattern observed in control subjects. The method of analysis typically measures the likelihood of a subject having TB.

The patients having TB have typically been diagnosed as having TB as a result of culture of Mycobacterium tuberculosis from a sample derived from each patient. The control subjects may be selected from one or more of patients with respiratory infections other than TB, patients with sarcoidosis, patients with inflammatory bowel disease, patients with malaria, patients with human African trypanosomiasis (HAT), patients with neurological diseases, patients with autoimmune disease, patients with melanoma and healthy subjects. Patients suffering from other diseases not listed above, which patients do not have TB may also be used as control subjects. Typically, the control subject expression data to which the expression pattern of markers from the test subjects are compared comprise at least two, for example at least three, at least four, at least five, at least six, at least seven or at least eight, of the above mentioned subjects. Patients who are HIV positive are particularly susceptible to disease. The TB patients and/or the control subjects may be HIV positive or HIV negative.

The TB and control samples may be taken from patients and/or subjects from more than one, for example, two or more, three or more, four or more, five or more, eight or more or ten or more, geographical sites. Each geographical site may be a different continent, country or region within a country. Different samples from TB and/or control subjects may be processed to obtain expression data at different times. For example, the samples may be obtained and/or processed over any suitable period of time, such as one month to two years, three months to eighteen months or six months to one year.

The method by which it is determined whether the expression data is indicative of TB, or not, is typically implemented using a computer. The computer may be physically separate from or may be coupled to the reader used to generate expression data, for example to the mass spectrometer.

Supervised machine learning classification methods may be used to discriminate the expression data of patients with TB from expression data of the control subjects. The machine learning classifier is first trained using training expression data from TB patients and training control data from the control subjects.

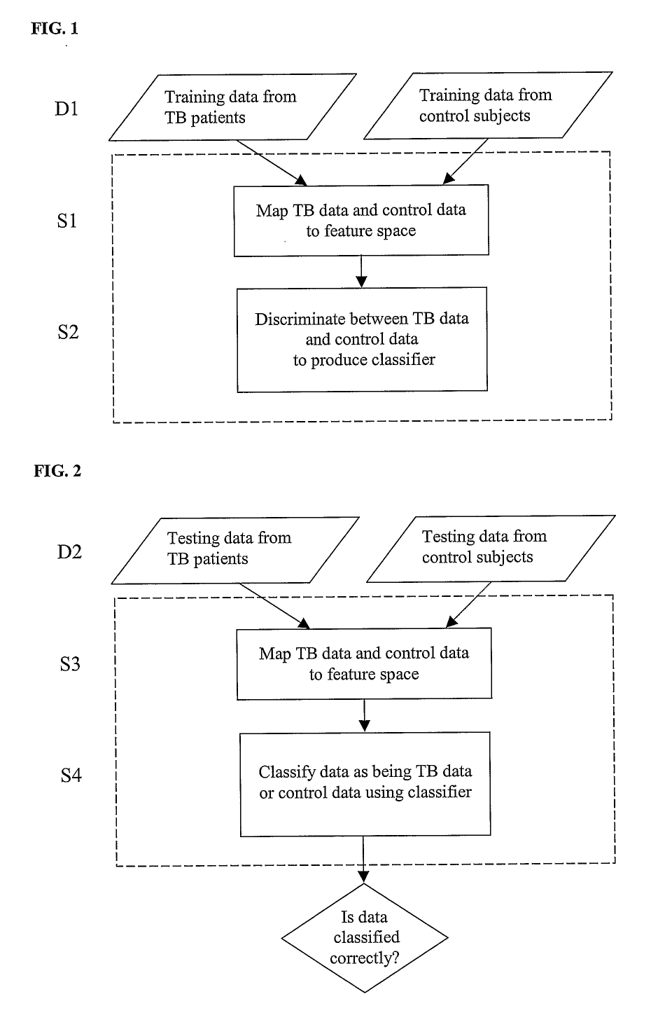

A method of training a machine learning classifier to distinguish expression data from a TB patient from expression data from a subject who does not have TB is illustrated in the flow chart of FIG. 1. The steps carried out by a computer program executed on a computer system are illustrated schematically by a dotted line in FIG. 1. The training data from TB patients and control objects (data D1) represent input variables (typically m/z values, ELISA values or nephelemetry values). In step S1, the computer maps these input variables to feature space using a kernel and in step S2 the classifier learns to discriminate between TB data and control data thus producing a training classifier, such as a SVM, to discriminate between TB data and control data.

The trained classifier may then be tested using expression data from further TB patients and further control subjects. A method of testing the generalisation of a machine learning classifier is illustrated in the flow chart of FIG. 2. The computer-implemented steps are illustrated schematically by a dotted line in FIG. 2. Independent training and testing sets may be used, with similar numbers of TB cases and controls and similar representation of age and sex in each set, for example as shown in Table 1. The testing data from TB patients and/or control subjects (data D2) represent input variables (typically m/z values, ELISA values or nephelemetry values). The computer maps these input variables to feature space using a kernel in step S3 and the classifier produced using training data is used in step S4 to assign the class of the input variables as being TB data or non-TB data. It can then be determined whether the test data has been classified correctly or mis-classified.

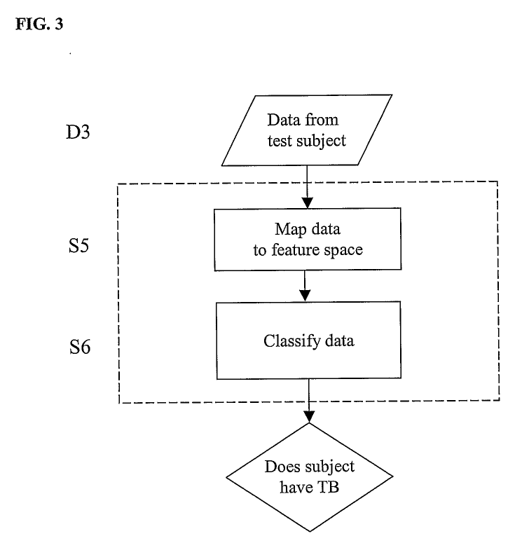

A trained machine learning classifier may be used to determine whether expression data from a subject whom it is wished to diagnose as having, or not having, TB is indicative of the patient having, or not having, TB. The trained machine learning classifier used in such a method of diagnosis may have been tested as described above, but this testing step is not essential. FIG. 3 is a flow chart which illustrates a computer-implemented method of diagnosis according to the invention. The computer-implemented steps are illustrated schematically in FIG. 3 by a dotted line. The data from the test subject (i.e. a new unknown subject) labelled D3 in FIG. 3 represents the input variables. In step S5, the computer maps the input variables (typically m/z values, ELISA values or nephelemetry values) to feature space using a kernel and the previously obtained classifier is used in step S6 to classify the sample as being a TB sample or non-TB sample. Hence, the test subject is diagnosed as having or not having TB.

Suitable machine learning classifiers include the single layer perceptron (SLP), the multi-layer perceptron (MLP), decision trees and support vector machines. Preferably the classifier in a support vector machine. More preferably, the classifier is a Gaussian kernel support vector machine.

A supervised leaning algorithm is tasked to find a decision function capable of assigning the correct label for a set of input/output pairs of examples, called the training data. The ability of the decision function to predict correct labels for unseen samples (test data) is known as its generalization. Current machine learning methods such as support vector machines (SVM) aim to optimize this property. The generalization of a classifier is dependent on a set of parameters (model) that must be chosen to optimise performance. For this purpose a grid search strategy may be adopted in which a range of parameter values are discretized and tested using cross-validation.

A dataset D is represented by a sample of input vectors, X, (i.e. exemplars of categories) with their corresponding sample of output labels, Y,D=[X,Y]. A sample input vector is represented by x. The mass spectrum of the i-th sample is represented as an n-dimensional (number of mass clusters) vector xᵢ with an associated class label yᵢ (+1 for TB, −1 for control) where i=1, . . . , m and m is the number of samples. The spectrum vector elements are denoted by x_{i,k} where i=1, . . . , m and k=1, . . . , n. The classifier prediction of a sample class label yᵢ is denoted by ŷᵢ.

The Support Vector Machine (SVM) maps its inputs to a high or even infinite dimensional feature space. The output of the SVM is then a linear thresholded function of the mapped inputs in the feature space, which may be nonlinear in the original input space. The mapping is accomplished by a user-selected reproducing kernel function K(x, x′) where x and x′ are input vectors. The kernel function must satisfy Mercer's conditions. Well-known examples of kernels include the Gaussian

$$K ( x , {x}^{′} ) = {e}^{- \frac{{‖ x - {x}^{′} ‖}^{2}}{2 {σ}^{2}}}$$

where the parameter σ determines the width; and the polynomial K(x, x′)=(x·x′)^{d} where d determines the degree. When d=1 it is called the linear kernel and corresponds to the identity map of the input data. A trained SVM classifier has the form

$$svm\_classifier ( x ) = sign ( {\sum }_{i = 1}^{m} {α}_{i} K ( {x}_{i} , x ) + b )$$

and training determines the values of a and b. Typically, many of the as will be zero. Those that are non-zero are called ‘support vectors’ and are used to define a separation hyperplane in the transformed feature space. Training a SVM is a convex (quadratic) optimization problem not subject to local minima unlike a multi-layer perceptron. There are many packages available to train an SVM; such as SVM^{light} (Joachims, 1999) and, in particular, soft-margin SVMs which are practicable when data are noisy. In this case the algorithm also minimizes the distance of incorrectly classified examples to the margin by adjusting a penalty value, C, called the soft-margin parameter.

The Single Layer Perceptron (SLP) (Rosenblatt, 1962) is an artificial neural network with one output neuron that computes a linear combination of the values given by the input layer. The discrimination function is given by

$$\hat{y} = sign ( {\sum }_{i = 1}^{n} {w}_{i} {x}_{i} + b )$$

where weights w are obtained by an iterative leaning
algorithm designed to reduce the total classification error

$${\sum }_{i = 1}^{m} \vert {y}_{i} - {\hat{y}}_{i} \vert .$$

The Multi-Layer Perceptron (MLP) (McClelland and Rumelhart, 1986) is a generalization of the SLP with intermediate layers of hidden neurons. It tackles the problem of non-linearly separable classes by allowing the neurons to process their inputs with a sigmoid function on the activation level

$$f ( a ) = \frac{1}{1 + {e}^{- a}} .$$

In this network the weights are learned by a back-propagation algorithm which is a gradient descent rule to minimize the error given by

$${\sum }_{i = 1}^{m} {( {y}_{i} - {\hat{y}}_{i} )}^{2} .$$

A decision tree learns to classify a dataset of samples D=[X,Y] by aggregating their features within a set of nodes organized in a binary tree structure. To find the tree structure, sample features are tested according to their discriminative power using a splitting criterion: for a given mass peak x_{i,k} the test x_{i,k}<T where T is any test that produces a binary partition of dataset D. In the C4.5 (Quinlan et al., 1993) classifier the test thresholds are evaluated by an information-gain splitting criterion

$$Gain ( D , T ) = Info ( D ) - {\sum }_{i = 1}^{z} \frac{\vert {D}_{i} \vert}{\vert D \vert} \times Info ( {D}_{i} )$$

where Info(D) is an entropy measure of the class to which the sample belongs and z is the number of outcomes of the test T. An iterative algorithm places nodes with increasing information gain from the root to the leaves of the tree. The final tree might be pruned in order to get a more compact representation of the classifier. A testing set sample can be classified by testing its mass peak values against those in the nodes of the tree following a path from the root to a leaf with a classification output. The C5.0 algorithm is an extended version of C4.5 that winnows irrelevant features and incorporates variable misclassification costs (http://www.rulequest.com/). The Alternating Decision Tree (ADTree) (Freund and Mason, 1999) is a tree with additional nodes for predicting values that are summed over a classification path and the final output is the sign of this sum.

Any suitable cross-validation scheme may be used such as k-fold cross-validation or k-fold cross-validation with test. In k-fold cross-validation the training set is randomly split in k groups of equally distributed positive and negative cases. A classifier is trained on k−1 of the groups and its generalization performance is validated on the remaining group. This process is repeated k times, each time holding out a different validation subset and the average represents the overall generalization. In the second scheme, k-fold cross-validation with test, the data is first randomly split into training and testing sets. A k-fold cross-validation is performed on the training set and the generalization is obtained on the unseen testing set.

The generalization performance of the classifiers may be assessed by considering the number of correctly classified (true positives, TP and true negatives, TN) and incorrectly classified (false positives, FP and false negatives, FN) cases in the testing set. Sensitivity (se), may be defined as the conditional probability of a true positive se=TP/(TP+FN), specificity (sp) as the conditional probability of a true negative sp=TN/(TN+FP), and accuracy (ac) as the proportion of correct classifications ac=(TP+TN)/(TP+FP+TN+FN). The performance of a classifier expressed by its true positive rate (se) and false positive rate (1−sp) can be plotted in a receiver operator curve (ROC) space.

Robust estimates of the generalization capability of the classifier may be provided by carrying out 10-fold cross-validation with test. For example, one hundred 80:20 train:test sets may be generated by random sampling without replacement in the entire dataset. For each 80:20 train:test set a 10-fold cross validation is carried out on the training set and the parameter with the best performance is chosen. The SVM may be re-trained with the best parameter over all the 10 subsets and the final performance is assessed on the testing set. Each ROC curve may be smoothed, sampled and averaged in order to show the mean curve with standard deviation.

The invention further provides a computer-implemented method of diagnosing TB, said method consisting essentially of the steps of:
(a) inputting expression data of two or more markers in a subject; and
(b) determining whether expression of said markers is indicative of TB using a computer system programmed with a trained support vector machine (SVM);
thereby diagnosing whether or not said patient has TB.

The expression data may relate to any two or more markers which are differentially expressed in TB patients and control subjects and include the markers described above. In one embodiment, the expression data is a proteomic profile from a sample from the subject, typically a blood, plasma or serum sample, obtained by SELDI analysis.

The support vector machine is trained as described above and is preferably a Gaussian kernel support vector machine. The computer system programmed with the trained support vector machine classifies the expression data from the subject as being indicative of the subject having TB, or of the subject not having TB. Accordingly, the output from the computer system enables diagnosis of the subject as having, or not having, TB.

Based on a diagnosis of TB by a method of the invention, further processes may be instigated. A method of diagnosis according to the invention may further comprise administering to a patient diagnosed as having TB, a medicament for the treatment of TB. A medicament for treating TB is a substance or composition that, when administered to a subject in a therapeutically effective amount, alleviates the symptoms or otherwise lessens the suffering of the subject. The substance or composition may be an agent which kills or disables Mycobacterium tuberculosis, for example by preventing its replication. Suitable medicaments include isoniazid, rifampin, pyrazinamide and ethambutol. The exact treatment regime may depend on the state of the individual, for example whether the individual is pregnant, HIV-seropositive, diabetic, etc and may readily be determined by a physician.

The present invention further provides a method of training a support vector machine (SVM) classifier to diagnose TB, said method consisting essentially of the steps of:
(a) providing training data which comprises:
   (i) training data relating to two or more markers in each of a first set of TB patients; and
   (ii) training data relating to said two or more markers in each of a first set of control subjects; and
(b) using a SVM to discriminate the training data of TB patients from the training data of control subjects;
thereby training the SVM to diagnose TB.

The method optionally further consists essentially of:
(c) providing testing data which comprises:
   (i) testing data relating to said two or more markers in each of a second set of TB patients; and
   (ii) testing data relating to said two or more markers in each of a second set of control subjects;
(d) determining the ability of the SVM to correctly discriminate the testing data of TB patients from the testing data of control subjects.

The training and testing data may be obtained by any suitable method, such as those described above.

The testing data is typically used to determine the sensitivity, specificity and/or accuracy of the SVM classifier.

The invention further provides an apparatus arranged to perform a method of diagnosis according to the invention, which apparatus consists essentially of,
(i) means for receiving expression data of two or more markers in a sample from a subject;
(ii) a module for determining whether said data is indicative of TB, wherein said module comprises a trained machine learning classifier capable of distinguishing data from a TB patient and data from a control subject; and
(iii) means for indicating the results of said determination.

The means for receiving expression data may be a keyboard into which data may be entered manually. Alternatively, the expression data may be received directly from the computer analysing the expression data, such as the protein chip reader or automated image analyser. The expression data may be received by a wire, or by a wireless connection. As a further alternative, the expression data may be recorded on a storage medium in a form readable by the apparatus. The storage medium may be placed in a suitable reader comprised within the apparatus.

The training, testing and or expression data from a subject being tested for TB may be raw data or may be processed prior to being inputted into the computer system. The computer system may comprise a means for converting raw data into a form suitable for further analysis.

The module for determining whether the data is indicative of TB, comprises a machine learning classifier which has been trained by a method as described herein such that it is able to distinguish expression data characteristic of a TB patient from expression data characteristic of a control subject.

The means for indicating the results of said determination may be a visual screen, audio output or printout. The results typically indicate the classification of the expression data and may optionally indicate a degree of certainty that the classification is correct.

The apparatus of the invention may be a personal computer. The personal computer may be a laptop. Alternatively, the apparatus may be a hand held computer, for example a specifically designed hand held computer, which has the advantage of being readily transportable in the field.

The invention further provides a computer program executable by a computer system, the computer program being capable, on execution by the computer system, of causing the computer system to perform a method of diagnosis according to the invention. The computer program generally comprises a machine learning classifier, preferably a support vector machine, which has been trained as described herein.

The invention further provides a storage medium storing in a form readable by a computer system a computer program of the invention. Any suitable storage medium may be used such as a CD-ROM or floppy disk.

In a further aspect, the invention provides a kit for use in the diagnosis of TB. The kit typically comprises means for detecting two or more markers as defined herein. The means of detection typically comprises a capture surface as described herein, such as a protein chip or array of specific binding reagents such as antibodies or antibody fragments. The kit may comprise instructions for operation in the form of a label or separate insert. For example, the instructions may inform a consumer how to collect the sample, how to incubate the sample with the capture surface and/or how to wash the probe. The kit may comprise instructions for inputting expression data of the markers into an apparatus of the invention. The kit may comprise a storage medium of the invention.

The kit is preferably adapted to detect any combination of two or more, such as three, four, five or six or more of the markers, transthyretin, neopterin, CRP, SAA, Apo-A1, serum albumin, Apo-A2, hemoglobin beta, haptoglobin protein, DEP domain protein, A2GL and hypothetical protein DFKZp6671032. In one preferred embodiment, the kit is adapted to detect any combination of two or more, such as three or four of the markers transthyretin, neopterin, CRP and SAA, for example, transthyretin, neopterin and CRP. The kit may be capable of detecting additional markers other than these four specified markers.

The kit may be adapted to detect the positive markers and/or negative markers set out in the Table below.

| Positively Correlated | Negatively Correlated |
|---|---|
| ‘M18394_9’ | ‘M4100_03’ |
| ‘M8952_75’ | ‘M3898_52’ |
| ‘M11720_0’ | ‘M13774_3’ |
| ‘M11454_1’ | ‘M13972_1’ |
| ‘M18591_2’ | ‘M3322_01’ |
| ‘M11488_1’ | ‘M2956_45’ |
| ‘M11541_5’ | ‘M5644_96’ |
| ‘M9076_68’ | ‘M3939_63’ |
| ‘M8895_13’ | ‘M4056_39’ |
| ‘M10856_8’ | ‘M6649_74’ |

In this embodiment, the detection means is preferably a protein chip.

The kit may additionally comprise one or more sample of one or more marker in a container. The marker provided in the kit may be used as a control or for calibration.

The invention also provides methods for identifying candidate agents for the treatment of TB. Candidate agents may be identified by assaying for activity of a test agent in modifying activity or expression of one or more of transthyretin, neopterin, CRP, SAA, serum albumin, Apo-A1, Apo-A2, hemoglobin beta, haptoglobin, DEP domain protein or A2GL. The biological activities of each of transthyretin, neopterin, CRP, SAA, serum albumin, Apo-A1, Apo-A2, hemoglobin beta, haptoglobin, DEP domain protein or A2GL are known in the art. Accordingly, the skilled person would readily be able to perform assays to assess the effect of a test agent on the activity of any one of transthyretin, neopterin, CRP, SAA, serum albumin, Apo-A1, Apo-A2, hemoglobin beta, haptoglobin, DEP domain protein or A2GL.

In one embodiment of the invention, candidate therapeutic agents may be identified by determining the effect of a test agent on the expression of one or more TB marker in cells infected with Mycobacterium tuberculosis. The one or more TB marker is generally selected from transthyretin, neopterin, CRP, SAA, serum albumin, Apo-A1, Apo-A2, hemoglobin beta, haptoglobin, DEP domain protein, A2GL and hypothetical protein DFKZp6671032. An increase or decrease in expression of one or more marker indicates that the test agent is useful in the treatment of TB. Typically, where the marker is a positive marker of TB, a test agent useful in treating TB reduces the level of expression of the marker compared to the level of expression in infected cells in the absence of the test agent. Typically, where the marker is a negative marker of TB, a test agent useful in treating TB increases the level of expression of the marker compared to the level of expression in infected cells in the absence of the test agent.

The infected cells may be in vivo or ex vivo. Where the cells are in vivo, they are typically present in an experimental animal, typically a rodent, such as a mouse or a rat. The infected cells may be any cells which Mycobacterium tuberculosis is capable of infecting. In one embodiment the cells are cells of the respiratory system, or cell lines derived therefrom.

Also provided by the invention are candidate therapeutic agents identified by such methods of the invention. Suitable candidate agents include antibodies specific for one of transthyretin, neopterin, CRP, SAA, serum albumin, Apo-A1, Apo-A2, hemoglobin beta, haptoglobin, DEP domain protein or A2GL.

The following Examples illustrate the invention.

### EXAMPLES

### Example 1

### Selection of Patients and Control Subjects

To develop new approaches for diagnosing TB we collected sera from cases (n=179) and controls (n=170) from multiple sites (UK, Angola, The Gambia and Uganda) representing patients from at least 4 ethnic backgrounds (Table 1). We confined ourselves to patients with TB who presented with typical manifestations of pulmonary disease (Rathman et al., 2003), because this is the commonest presentation of adult TB in all geographic areas. Diagnosis was confirmed by culture of M. tuberculosis. Details of patients that include both smear positive and smear negative cases, and control subjects (including HIV status) are given in Tables 1 and 2a. As expected, most patients presented with cough, fever and weight loss, and the majority had cavitary pulmonary disease.

For our control subjects, we recruited healthy volunteers as well as patients having conditions with clinical features that can overlap with TB (Table 2b). Our control subjects have heterogeneous causes of inflammation that have been confirmed by standard diagnostic criteria. For example, we included patients with sarcoidosis, which is frequently included in the differential diagnosis of pulmonary TB, and other severe respiratory infections representing patients who have non-tuberculous destructive pulmonary pathology. To allow for systemic inflammatory processes that can mimic TB, we recruited patients with other systemic infections as well as patients with inflammatory bowel and autoimmune diseases.

### Example 2

### Proteomic Profiling and Supervised Machine Learning Classification

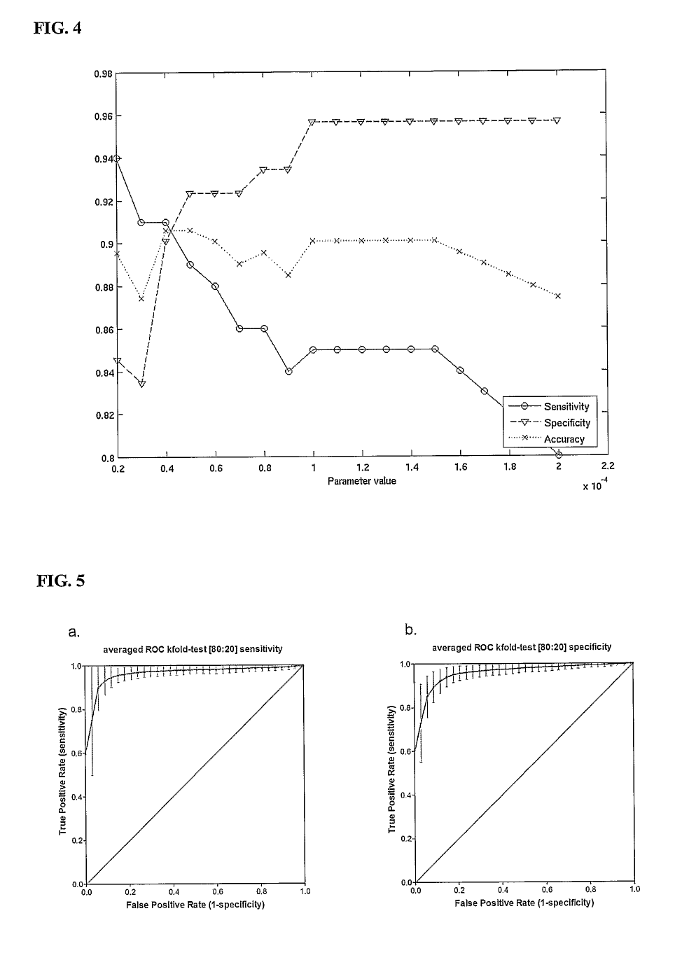

We first profiled 349 serum samples from these subjects on weak cation exchange (CM10) protein chip arrays by Surface Enhanced Laser Desorption lonisation Time of Flight Mass Spectrometry (SELDI-TOF MS) (Issaq et al., 2002; von Eggeling et al. 2001) and identified 219 peak clusters from m/z spectra in the range 2,000-100,000. We then used state-of the-art supervised machine learning classification methods (Table 3 and FIG. 4) to discriminate the proteomic spectra of patients with TB from the controls using the training-testing-set approach (Table 1). The ability of a classifier to correctly discriminate data in the testing set is known as its generalization performance (Vapnik, 1998; Cristianini and Shawe-Taylor, 2000). We compared the generalization performance of a variety of classifiers by plotting their performance on such a testing set in Receiver Operating Characteristic (ROC) space.

In our study the SLP did not provide an optimal discriminative function, giving an accuracy of 86.5% in the independent test set (Table 3). With our data the MLP showed similar generalization performance to SLP, classifying with an accuracy of 86.5% (Table 3). In the TB versus control dataset (Table 2) the ADTree and the C4.5 classifiers achieved accuracies of 92.3% and 91.0% respectively (Table 3), but relied on AdaBoost boosting to achieve such levels of generalization (Witten and Frank, 2000) (Table 3). We used AdaBoost with 100 iterations for the ADTree and C4.5 classifiers, and boosting with a maximum of 10 iterations for the non-commercial version of the C5.0 classifier.

A Gaussian kernel support vector machine (Boser et al., 1992; Vapnik, 1998; Cristianini and Shawe-Taylor, 2000) (SVM, Table 3) is the best discriminator between TB and control groups, having a sensitivity of 93.5% and a specificity of 94.9% (overall accuracy 94.2%). Five TB samples and 4 controls in the testing set were misclassified. This SVM classifier defines the convex hull of the ROC space achieving the best accuracy.

We applied a further test of generalization performance of the SVM by carrying out 10-fold cross-validation on the entire set of spectra (both training and testing), obtaining accuracy of 93.1±3.8%, sensitivity of 94.4±4.5% and specificity of 91.8±8.8% when optimised for accuracy. We also evaluated the generalisation performance of the SVM by varying the proportions of train:test cases from 90:10 to 50:50. For 80:20 sets, we obtained values for accuracy, sensitivity and specificity exceeding 90%. The robustness of the SVM is further confirmed by its mean performance on 100 randomly generated 80:20 sets as shown in the ROC curve, with an area under the curve (AUC) of 0.96. FIG. 5 shows the averaged ROC using the 10-fold train cross validation test. In FIG. 5a the kernel parameter is selected on sensitivity only and in FIG. 5b the kernel parameter is selected on specificity criteria.

In spite of the deliberate heterogeneity of the control group, our classifier discriminates accurately between patients with TB (both smear negative and smear positive) and those with a range of infective and non-infective inflammatory conditions. These results show that TB is amenable to a proteomic-signature based diagnostic approach. Artefacts associated with sample collection, handling or spectrum generation could potentially create spurious classifications. However, interspersing the processing of samples from TB cases and control subjects over a 6 month period and using samples from 4 different geographic sites and varying HIV sero-status, makes systematic biases between cases and control subjects highly unlikely. As a measure of reproducibility of the mass spectra, 28 universal control spectra run at different times over a 6 month period were correctly classified as control subjects by the SVM classifier obtained in the 10-fold cross-validation. In a clinic population where the prevalence of TB in patients presenting with respiratory symptoms is around 10%, the positive and negative predictive values for our best classifier would be 67% and 99% respectively. This diagnostic accuracy surpasses that of other available diagnostic options.

### Example 3

### Selection of Markers

However, while SELDI technology can provide a diagnostic test for TB that makes no prior assumptions about the identities of proteins constituting an informative signature, cost and complexity may preclude its widespread general use. We therefore selected a subset of informative peak clusters for further evaluation by applying a correlation filter method to detect independently informative peaks (Guyon and Eliseeff, 2003). We ranked 10 mass clusters with the highest positive, and 10 with the highest negative, Pearson correlation coefficients. The m/z values of these markers is shown in the Table below.

| Positively Correlated | Negatively Correlated |
|---|---|
| ‘M18394_9’ | ‘M4100_03’ |
| ‘M8952_75’ | ‘M3898_52’ |
| ‘M11720_0’ | ‘M13774_3’ |
| ‘M11454_1’ | ‘M13972_1’ |
| ‘M18591_2’ | ‘M3322_01’ |
| ‘M11488_1’ | ‘M2956_45’ |
| ‘M11541_5’ | ‘M5644_96’ |
| ‘M9076_68’ | ‘M3939_63’ |
| ‘M8895_13’ | ‘M4056_39’ |
| ‘M10856_8’ | ‘M6649_74’ |

To study the discriminatory power of the selected 20 mass clusters we first paired each mass with every other (400 pairs) and trained SVM classifiers to diagnose TB cases. The results are shown in Table 4. We ranked generalization performance by accuracy and showed that 20 pairs (5%) of selected mass clusters gave accuracies greater than 80% and 17 of these combined negatively-correlated and positively-correlated mass clusters. No mass cluster pair achieved sensitivities and specificities greater than 95% and 85%, respectively, confirming that better generalization relies on combinations of more than two mass peaks. Second, an SVM trained with just the 20 correlation-selected mass clusters achieved an accuracy of 89.7% on the independent test set indicating that these clusters contain most relevant discriminatory information. Information in remaining peak clusters (n=199) retains an inferior though acceptable diagnostic accuracy (85.9%). We summarised the generalization performance of the SVMs in ROC space using different sets of mass clusters. The ROC convex hull is defined by 2 classifiers. The highest specificity was obtained with all peaks minus the 10 that were positively correlated (i.e. 209 in total), confirming information value in negatively correlated peaks. The other optimal classifier was obtained after using only 10 positively and 10 negatively correlated subsets of mass clusters.

### Example 4

### Identification of Markers

Using high-resolution mass-spectrometry after tryptic digestion we identified an 11.5 kDa ‘positive’ marker and a 13.7 kDa ‘negative’ marker as the des-arginine variant of serum amyloid A1 (SAA1) and transthyretin, respectively. Interestingly, these peptides, selected by Pearson correlation analysis and confirmed by SVM classification of proteomic signatures, have already been independently associated with pathophysiological processes in TB. SAA is an acute phase protein associated with circulating high-density lipoprotein (HDL) (Kieman et al., 2003) and modulating lipid trafficking and immune responses. It is the precursor protein in reactive amyloidosis, which complicates chronic TB in some individuals, and is a marker of disease activity in several inflammatory states including tuberculosis (Salazar et al., 2001). Transthyretin is a 55 kDa homotetramer in serum and a major transporter of thyroxine and tri-iodothyronine, as well as vitamin A (retinol or trans-retinoic acid) through association with retinol-binding protein (Peterson, 1971). Retinoic acid stimulates monocyte differentiation and inhibits multiplication of M. tuberculosis in human macrophages (Crowle et al., 1989). Low levels of vitamin A, correlating with reduced transthyretin and elevated C-reactive protein levels, have been reported in patients with TB (Hanekom et al., 1997; Koyanagi et al., 2004).

### Example 5

### Immunoassay Tests and Supervised Machine Learning Classification

To translate from proteomic signatures to conventional test formats, we quantitated serum SAA and transthyretin by immunoassay in all subjects. Because both peptides are markers of inflammation, we also measured C-reactive protein (CRP) and neopterin that have previously been used to monitor disease activity in TB (Hosp et al., 1997). We then parameterised polynomial and Gaussian kernel SVMs for these 4 markers. The best 4 classifiers were obtained using Gaussian SVMs. The SVM classifier trained with transthyretin, CRP and neopterin values discriminated TB from control patients with an accuracy of 84% (82% sensitivity, 86% specificity). Other optimised classifiers were with SAA and CRP with transthyretin included, and using transthyretin and neopterin. Inclusion of additional markers in the original signature is likely to improve accuracy of immunoassay-based classifications.

A truncated form of transthyretin is a negative marker in proteomic fingerprinting studies on ovarian cancer (Zhang et al., 2004) and SAA is a positive marker in Severe Acute Respiratory Syndrome (SARS) (Ren et al, 2004) and indicates relapse in nasopharyngeal cancer (Cho et al., 2004). Although single protein markers may have insufficient accuracy in the diagnosis of TB, the use of proteome-guided analysis coupled with machine learning methods such as SVM can achieve accuracies that are superior to current standard methods. These findings suggest that markers with low individual diagnostic specificities can boost diagnostic yields when used in particular combinations. In some cases, truncated or fragmented derivatives of common plasma proteins may be more specific markers of some diseases and arise by proteolytic enzyme induction characteristic of defined disease states (Tolson et al., 2004). Preservation of high diagnostic accuracy when translating from proteomic signatures to immunoassays, and the biological plausibility of identified biomarkers establishes the value of SVM classifiers for diagnosis of TB and provides strong foundations for serological testing. Provision of trained SVM classifiers on personal computers provides an opportunity to aid TB diagnosis using immunoassays (or where available, SELDI proteomic analysis). These tests can then be applied to longitudinal studies of TB and other difficult diagnostic categories such as patients with sputum negative TB, extra-pulmonary cases and paediatric infections.

### Example 6

### Materials and Methods

Serum collection and storage. Serum samples (179) were collected from patients with retrospectively confirmed culture-positive TB (Table 2). Banked sera collected in Uganda and The Gambia were obtained from the World Health Organisation TB specimen bank (http://www.who.int/tdr/diseases/tb/specimen.html), and others were collected prospectively from patients presenting with TB to the inpatient and outpatient facilities at St George's Hospital, London, UK. Serum samples (170) from control patients with a range of other inflammatory conditions were collected at St George's Hospital, UK, the Angotrip treatment centre, Angola and The Gambia. Fully informed consent was obtained in each case, in accordance with local Research Ethical Committee policy. Clinical information was archived in a linked, anonymised database. Serum was separated from 5 ml blood by centrifugation, and samples allowed to clot for 30 minutes at room temperature in sterile glass tubes. Aliquots (100 μl) were frozen (−80° C.) within 1 hour of collection, and subjected to no more than two freeze-thaw cycles prior to mass spectrum analysis.

Sample preparation for mass spectrometry. Samples were applied to CM10 protein chip arrays (Ciphergen, Fremont, Calif., USA) as described previously (Papadopoulos et al., 2004), and a saturated solution of sinapinic acid in 50% acetonitrile, 0.5% triflouroacetic acid was applied twice to each spot on the array, with air drying between each application. To minimise bias, sera from TB patients and controls were assayed on the same chips.

Surface Enhanced laser Desorption lonisation Time of Flight Mass Spectrometry (SELDI-ToF MS). Time-of-flight spectra were generated using a PBS-II Mass spectrometer (Ciphergen, Freemont, Calif., USA) at laser intensities of 200, 220 and 240, high mass 100 kDa, detector sensitivity 8 and focus mass 10 kDa. Each spot on the array was analysed from position 20 to 80, delta 4, with 7 shots per position, preceded by 2 warning shots at laser intensities of 205, 225 or 245. Each protein chip array included a ‘universal control’ sample (aliquoted from a single collection from one individual and stored at −80° C.). Both groups of spectra (TB and controls) comprised samples run on different occasions over a 6 month period.

Peak identification. Spectra were calibrated weekly using the Ciphergen all-in-one protein and peptide calibrants, and normalised to the total ion current in the m/z range over 2,000-100,000 after baseline subtraction. For each patient a single spectrum generated at a laser intensity of 200, 220 or 240 was selected to minimise deviation of the total ion current to within 0.4-2.6 times the mean of all patients as described previously (Papadopoulos et al., 2004). Biomarker Wizard version 3.1 was used to identify corresponding peaks in each spectrum (‘peak clusters’) within 0.6% of the molecular mass. Signal-to-noise ratio was set at 10 for the first pass and 2 for the second pass. To assess reproducibility, coefficients of variation for peak size for spectra derived from a single sample run 25 times (6 assays) were 15.6% (intra-assay) and 24.4% (inter-assay). These data were obtained by averaging values for 9 of the highest amplitude peaks at the following m/z values: 5648, 6203, 6449, 6647, 8907, 9213, 9310, 9370 and 9419.

Protein identification. Serum (20 μl) was incubated on ice (20 minutes) with 30 μl denaturation buffer, diluted in 50 μl binding buffer (denaturation buffer diluted 1:9 in 50 mM Tris-HCl pH9.0) followed by a further 30 minute incubation on ice. Samples were applied to Q Ceramic HyperD spin columns (Ciphergen, 20 minutes), pre-equilibrated first in Tris (50 mM, pH 9), followed by binding buffer. Both the 11.5 kDa and 13.7 kDa biomarkers were eluted from the spin column in elution buffer (50 mM Na citrate, 0.1% octyl glucopyranoside, pH 3) and selective enrichment was confirmed by SELDI-ToF MS analysis of a sample of eluate applied to a CM10 protein chip array under conditions as described above for unfractionated serum.

The biomarkers were isolated by 1D SDS-PAGE (NuPAGE, 4-12% Bis-Tris, Invitrogen), stained with Coomassie Blue and excised from the gel. The gel pieces were washed three times in a mixture of ammonium bicarbonate (50 mM) and acetonitrile (50%), dehydrated in acetonitrile (100%) and dried.

Proteins were subjected to in-gel tryptic digestion (15 minutes, RT) by the addition of trypsin (20 ng/μl) in acetonitrile (10%) and ammonium bicarbonate (25 mM), followed by a final incubation in ammonium bicarbonate (25 mM) for 4 hours.

Peptide mass fingerprints (PMFs) of the digests were analysed by MALDI-ToF MS using 20% α-cyano-4-hydroxy-cinnamic acid (CHCA) as matrix. The results of the in-gel tryptic digest were corroborated by tryptic digestion following passive elution of the protein from the gel.

The PMFs were used to interrogate the MASCOT database which identified the peptides as having been derived, in one case from serum amyloid A1 (SAA1) and in the other, from transthyretin. The molecular weight observed in the mass spectrum (13.7 kDa) for the protein identified as transthyretin corresponded closely to the theoretical value (13.76 kDa) of this protein. However that observed for SAA1 (11.52 kDa) was 156 Da lower than its theoretical value (11.68 kDa) suggesting that the protein was a SAA1 variant.

In order to investigate the nature of this valiant, the tryptic digest was analysed in more detail and found to include a peptide at m/z 1551 that did not correspond to a tryptic peptide predicted from the full amino acid sequence of SAA1. It did, however, correspond to the 2-15 peptide (SFFSFLGEAFDGAR) which would have resulted from loss of the N-terminal arginine.

Immunoquantitation of biomarkers. The lower limit detection for each marker and the antibody type used for detection were as follows: 0.7 mg/l SAA with particle enhanced sheep anti-SAA, 1 mg/l CRP with goat anti-CRP, 0.05 g/l transthyretin with goat anti-transthyretin and 1.5 nmol/l neopterin with rabbit anti-neopterin. Neopterin was measured by competitive ELISA using a kit (ELItest Neopterin, B.R.A.H.M.S Aktiengesellschaft, Germany) in a Triturus analyser (Grifols UK Ltd). Rate nephelemetry was used for measurement of C-reactive protein, transthyretin (Beckmann Image 800 analyser, Beckman Coulter UK, Ltd) and serum amyloid A (N latex SAA, BN II analyser, Dade-Behring, Marburg, Germany). The antibody used in the SAA assay detects total SAA. Values from ELISAs were scaled in the range 0-1 before use in SVM classification experiments, and all possible combinations were used as feature space.

Supervised Machine Learning. A dataset D is represented by a sample of input vectors, X, (i.e. exemplars of categories) with their corresponding sample of output labels, Y, D=[X,Y]. A sample input vector is represented by x. The mass spectrum of the i-th sample is represented as an n-dimensional (number of mass clusters) vector xᵢ with an associated class label yᵢ (+1 for TB, −1 for control) where i=1, . . . , m and m is the number of samples. The spectrum vector elements are denoted by x_{i,k} where i=1, . . . , m and k=1, . . . , n. The classifier prediction of a sample class label yᵢ is denoted by ŷᵢ.

A supervised learning algorithm is tasked to find a decision function capable of assigning the correct label for a set of input/output pairs of examples, called the training data. The ability of the decision function to predict correct labels for unseen samples (test data) is know as its generalization. Current machine learning methods such as SVM aim to optimize this property. The generalization of a classifier is dependent on a set of parameters (model) that must be chosen to optimise performance. For this purpose we adopted a grid search strategy in which a range of parameters values are discretized and tested using cross-validation.

The Support Vector Machine (SVM) maps its inputs to a high or even infinite dimensional feature space (Vapnik et al., 1998; Aronszajn, 1950). The output of the SVM is then a linear thresholded function of the mapped inputs in the feature space, which may be nonlinear in the original input space. The mapping is accomplished by a user-selected reproducing kernel function K(x, x′) where x and x′ are input vectors. The kernel function must satisfy Mercer's conditions (Joachims, 1999). Well-known examples of kernels include the Gaussian

$$K ( x , {x}^{′} ) = {e}^{- \frac{{‖ x - {x}^{′} ‖}^{2}}{2 {σ}^{2}}}$$

where the parameter a determines the width; and the polynomial K(x, x′)=(x·x′)^{d} where d determines the degree. When d=1 it is called the linear kernel and corresponds to the identity map of the input data. A trained SVM classifier has the form

$$svm\_classifier ( x ) = sign ( {\sum }_{i = 1}^{m} {α}_{i} K ( {x}_{i} , x ) + b )$$

and training determines the values of a and b. Typically, many of the ds will be zero. Those that are non-zero are called ‘support vectors’ and are used to define a separation hyperplane in the transformed feature space. Training a SVM is a convex (quadratic) optimization problem not subject to local minima unlike a multi-layer perceptron. There are many packages available to train an SVM; we used SVM^{light} (Rosenblatt, 1962) and in particular we trained soft-margin SVMs which are practicable when data are noisy. In this case the algorithm also minimizes the distance of incorrectly classified examples to the margin by adjusting a penalty value, C, called the soft-margin parameter.

We used two cross-validation schemes. In k-fold cross-validation the training set is randomly split in k groups of equally distributed positive and negative cases. A classifier is trained on k−1 of the groups and its generalization performance is validated on the remaining group. This process is repeated k times, each time holding out a different validation subset and the average represents the overall generalization. In the second scheme, k-fold cross-validation with test, the data is first randomly split into training and testing sets. A k-fold cross-validation is performed on the training set and the generalization is obtained on the unseen testing set.

The generalization performance of the classifiers was assessed by considering the number of correctly classified (true positives, TP and true negatives, TN) and incorrectly classified (false positives, FP and false negatives, FN) cases in the testing set. Sensitivity (se), was defined as the conditional probability of a true positive se=TP/(TP+FN), specificity (sp) as the conditional probability of a true negative sp=TN/(TN+FP), and accuracy (ac) as the proportion of correct classifications ac=(TP+TN)/(TP+FP+TN+FN). The performance of a classifier expressed by its true positive rate (se) and false positive rate (1−sp) can be plotted in a receiver operator curve (ROC) space.

We created independent training and testing sets, with similar numbers of TB cases and controls and similar representation of age and sex in each set (Table 1). Using these sets we evaluated the generalization performance of several supervised machine learning methods such as single layer perceptron (SLP) (McClelland and Rumelhart, 1986), multi layered perceptron (MLP) (Quinlan et al., 1993), tree classifiers (Freund and Mason, 1999; Freund and Schapire, 1996 and Witten and Frank, 2000) and support vector machines (Table 3).

To provide robust estimates of the generalization capability of the classifier we carried out 10-fold cross-validation with test. First, we generated one hundred 80:20 train:test sets by random sampling without replacement in the entire dataset. For each 80:20 train:test set a 10-fold c.v. is carried out on the training set and the parameter with the best performance is chosen. The SVM is re-trained with the best parameter over all the 10 subsets and the final performance is assessed on the testing set. In these experiments each ROC curve is smoothed, sampled and averaged in order to show the mean curve with standard deviation.

Mass peak cluster selection. We used the Pearson correlation coefficient to rank peaks for their discriminatory power. The Pearson correlation coefficient is defined as

$$R ( k ) = \frac{covariance ( {X}_{k} , Y )}{\sqrt{variance ( {X}_{k} ) variance ( Y )}}$$

where Xₖ is the random variable corresponding to the k^{th} component of sample input vectors x and Y is the random variable of output labels.

The estimate of R(k) is given by

$$\hat{R} ( k ) = \frac{{\sum }_{i = 1}^{m} ( {x}_{i , k} - {\overline{x}}_{k} ) ( {y}_{i} - \overline{y} )}{\sqrt{{\sum }_{i = 1}^{m} {( {x}_{i , k} - {\overline{x}}_{k} )}^{2} {\sum }_{i = 1}^{m} {( {y}_{i} - \overline{y} )}^{2}}}$$

where x_{i,k} correspond to value m/z of the mass cluster k of sample i, yᵢ is the class label for sample i and m is the number of samples. R(i) may be used a test statistic to assess the significance of a variable and it is linked to the t-test. We calculated \hat{R}(k) between values of each mass cluster and corresponding class labels across the training set (Table 1). We then used \hat{R}(k) to rank positively and negatively correlated mass clusters. Using this approach we selected 10 mass clusters with the highest positive, and 10 with the highest negative, correlation coefficients. The decision boundary found by the classifier and discriminating mass cluster pairs in the feature space induced by the kernel is shown in FIG. 2a (green lines).

Software. We used a chunking and decomposition implementation of the support vector machine SVM^{light}. We used Waikato Environment for Knowledge Analysis (WEKA) for decision tree algorithms, boosting and MLP. Experimentation framework was coded in MATLAB and Java. A custom and reusable object-oriented database was created using ObjectDB and interfaced with experimentation framework. The MATLAB interface to SVM^{light} was obtained from http://www.igi.tugraz.at/aschwaig/software.html.

### Example 7

### Assignment of Identities to Markers Identified by SELDI-ToF/MS

In order to assign identities to the protein biomarkers identified by SELDI-T of/MS as being capable of discriminating sera from patients with Tuberculosis from sera from normal individuals, a pool of sera from 20 patients with TB and a second pool of sera from 20 healthy controls were generated. These were separated by 2D gel electrophoresis. To match the SELDI peak mass of a biomarker to the mass of a protein spot within the 2D gel, a second 2D gel was run where each spot was excised and the protein eluted passively from it to generate a solution of the full length protein. The solution of full length protein was analysed by SELDI-T of/MS to generate a spectrum with a single peak. This mass was then compared with the original SELDI-T of/MS biomarker mass list. A match between the two SELDI-ToF masses identifies the gel spot as the one corresponding to the SELDI-T of/MS biomarker peak.

The gel spots from the matching 2D gel were removed and in-gel digested with trypsin to produce a peptide mixture diagnostic for that protein. This mixture was then analysed by LC/MS/MS to give a high probability prediction of identity based upon a BLAST search of the genome database.

Three biomarkers have been definitively identified in this way as shown in Table 5. The TB marker having an m/z value of 18394 is a serum albumin precursor, the TB marker having an m/z value of 11454 is Apo-A1 and the TB marker having an m/z value of 13774 is transthyretin.

### Example 8

### Identification of Further Markers

Analysis of the 2D gels containing serum proteins from TB patients and control subjects revealed that some proteins which did not appear to correspond to the markers identified by SELDI-ToF were differentially present in TB sera and sera from control subjects. The proteins were identified by removing the protein spots and in-gel digestion with trypsin to produce a peptide mixture diagnostic for that protein. The mixture was then analysed by LC/MS/MS to give a high probability prediction of identity based upon a BLAST search of the genome database. The additional markers identified were apolipoprotein-A2, hemoglobin beta, haptoglobin protein, DEP domain protein, leucine-rich-alpha-2-glycoprotein (A2GL or LRG1) and hypothetical protein DFKZp6671032.

The results of this analysis are shown in Table 6. As can be seen from Table 6, transthyretin was identified from both the control gel and the TB gel. However, transthyretin was expressed at a lower level in the TB gel compared to the control gel, confirming that transthyretin is a negative marker of TB. Similarly, Apo-A2 expression is lower in the TB gel compared to the control gel and so Apo-A2 is negative marker of TB. Similarly, haptoglobin and hemoglobin beta are both expressed at a lower level in the TB gel compared to the control gel and so are negative markers of TB. A2GL (LRG1) and DEP domain protein, on the other hand, are upregulated in the TB gel compared to the control gel and so are positive markers of TB.

Hypothetical protein DFI<Zp6671032 was found only in the control gel and so is a negative marker of TB.

### REFERENCES

Aronszajn, N. Theory of reproducing kernels. Trans Amer Math Soc 68, 337-404 (1950).
Boser, B. E., Guyon, I. M. & Vapnik, V. N. A training algorithm for optimal margin classifiers. in Proceedings of the fifth annual workshop on Computational Learning Theory 144-152 (Pittsburgh, Pa., United States, 1992).
Cho, W. C. S. et al. Identification of serum Amyloid A protein as a potentially useful biomarker to monitor relapse of nasopharyngeal cancer by serum proteomic profiling. Clin Canc Res 10, 43-52 (2004).
Cristianini, N. & Shawe-Taylor, J. An Introduction to Support Vector Machines and other kernel-based learning methods, (Cambridge University Press, Cambridge, 2000).
Crowle, A. J. & Ross, E. J. Inhibition by retinoic acid of multiplication of virulent tubercle bacilli in cultured macrophages. Infect Immun 57, 840-844 (1989).
Freund, Y. & Mason, L. The alternating decision tree learning algorithm. in In Proceedings of the Sixteenth International Conference on Machine Learning 124-133 (1999).
Freund, Y. & Schapire, R. E. Experiments with a New Boosting Algorithm. in Thirteenth International Conference on Machine Learning 148-156 (Morgan Kaufmann, Bari, Italy, 1996).
Guyon, I. & Eliseeff, A. An introduction to Variable and Feature Selection. J Machine Learn. Res 3, 1157-1182 (2003).
Hanekom, W. A. et al. Vitamin A status and therapy in childhood pulmonary tuberculosis. J. Pediatr. 131, 925-927 (1997).
Hosp, M. et al. Neopterin, beta 2-microglobulin and acute phase proteins in HIV-1-seropositive and -seronegative Zambian patients with tuberculosis. Lung 175, 265-275 (1997).
Issaq, H. J., Veenstra, T. D., Conrads, T. P. & Felschow, D. The SELDI-ToF MS approach to proteomics: protein profiling and biomarker identification. Biochemical and Biophysical Research Communications 292, 587-592 (2002).
Joachims, T. Making Large-Scale SVM Learning Practical. in Advances in Kernel Methods—Support Vector Learning (MIT Press, 1999).
Kiernan, U. A., Tubbs, K. A., Nedelkov, D., Niederkofler, E. E. & Nelson, R. W. Detection of novel truncated forms of human serum amyloid A protein in human plasma. FEBS Letts 537, 166-170 (2003).
Koyanagi, A., Kuffo, D., Gresely, L., Shenkin, A. & Cuevas, L. E. Relationships between serum concentrations of C-reactive protein and micronutrients in patients with tuberculosis. Ann Trop Med Parasitol 98, 391-399 (2004).
Maddox et al., J. Exp. Med. 158:1211-1226 (1993).
McClelland, J. L. & Rumelhart, D. E. Parallel and Distributed Processing, (MIT Bradford Press, 1986).
Papadopoulos, M. C. et al. A novel and accurate test for Human African Trypanosomiasis. Lancet 363, 1358-1363 (2004).
Peterson, P. A. Charactersitics of a vitamin A-transporting protein complex occurring in human serum. J. Biol. Chem 246, 34-43 (1971).
Quinlan, J. R. C4.5: Programs for Machine Learning, (Morgan Kaufmann, San Francisco, 1993).
Rathman, G. et al. Clinical and radiological presentation of 340 adults with smear-positive tuberculosis in The Gambia. Int J tuberc Lung Dis 7, 942-947 (2003).
Ren, Y. et al. The use of proteomics in the discovery of serum biomarkers from patients with severe acute respiratory syndrome. Proteomics 4, 3477-3484 (2004).
Rosenblatt, F. Principles of Neurodynamics, (Spartan Books, New York, 1962).
Salazar, A., Pinto, X. & Mana, J. Serum amyloid A and high-density lipoprotein cholesterol: serum markers of inflammation in sarcoidosis and other systemic disorders. Eur J Clin Invest 31, 1070-1077 (2001).
Tolson, J. et al. Serum protein profiling by SELDI mass spectrometry: detection of multiple variants of serum amyloid alpha in renal cancer patients. Lab Invest 84, 845-856 (2004).
Vapnik, V. Statistical Learning Theory, (John Wiley & Sons Inc, 1998).
von Eggeling, F. et al. Mass spectrometry meets chip technology: a new proteomic tool in cancer research? Electrophoresis 22, 2898-2902 (2001).
Witten, I. H. & Frank, E. Data Mining: Practical machine learning tools with Java implementations, (Morgan Kaufmann, San Francisco, 2000). Zhang, Z. et al. Three biomarkers identified from serum proteomic analysis for the detection of early stage ovarian cancer. Cancer Res 64, 5882-5890 (2004).

TABLE 1
Participant demographics

| | TUBERCULOSIS¹ | | | CONTROLS | | | |
|---|---|---|---|---|---|---|---|
| | Train | Test | Total | Train | Test | Total | TOTAL |
| Total no. of patients (%)² | 102 | 77 | 179 | 91 | 79 | 170 | 349 |
| Age (years) [mean (range)] | 31 (16-86) | 33 (19-84) | 32 (16-86) | 44 (16-88) | 46 (14-84) | 45 (16-84) | 38 (14-88) |
| Sex [male:female] | 65:37 | 47:30 | 112:67 | 52:39 | 42:37 | 94:76 | 206:143 |
| Ethnic Origin (%): | | | | | | | |
| Sub-Saharan African | 81 (79.4) | 60 (77.9) | 141 (78.8) | 28 (30.7) | 21 (26.5) | 49 (28.8) | 110 |
| African not specified | 3 (2.9) | 1 (1.3) | 4 (2.2) | 3 (3.3) | 3 (3.8) | 6 (3.5) | 90 |
| Asian | 13 (12.7) | 9 (11.6) | 22 (12.3) | 3 (3.3) | 0 | 3 (1.7) | 25 |
| White Caucasian | 5 (4.9) | 7 (9) | 12 (6.7) | 35 (38.4) | 29 (36.7) | 64 (37.6) | 76 |
| Not recorded | 0 | 0 | 0 | 22 | 26 | 48 | 48 |
| Collection Site: | | | | | | | |
| Uganda | 80 (78.4) | 59 (76.6) | 139 (77.6) | 0 | 0 | 0 | 139 |
| The Gambia | 1 (0.9) | 1 (1.3) | 2 (1.1) | 11 (12) | 10 (12.6) | 21 (12.3) | 23 |
| Angola | 0 | 0 | 0 | 10 (10.9) | 9 (11.3) | 19 (11.1) | 19 |
| UK (SGH) | 21 (20.5) | 17 (22) | 38 (21.2) | 70 (76.9) | 60 (75.9) | 130 (76.4) | 168 |
| HIV serology: | | | | | | | |
| HIV positive (%) | 35 (34.3) | 24 (31.1) | 59 (32.9) | 2 (2.2) | 3 (3.8) | 5 (2.9) | 64 |
| CD4 count ≧200 × 10⁶/ml (%)³ | 19 (54.3) | 13 (54.2) | 32 (54.2) | | | | |
| CD4 count <200 × 10⁶/ml (%) | 15 (42.8) | 11 (45.8) | 26 (44.1) | | | | |
| HIV negative (%) | 60 (58.8) | 45 (58.4) | 105 (58.6) | 12 (13.2) | 8 (10.1) | 20 (11.8) | 125 |
| HIV not determined (%) | 7 (6.8) | 8 (10.3) | 15 (8.3) | 77 (84.6) | 68 (86) | 145 (85.2) | 160 |

¹12 TB patients had received between 1 and 7 days of chemotherapy at time of recruitment to the study.
²Demographic data were missing for 24 patients in the training set and 25 in the testing set.
³CD4 counts were available for HIV seropositive patients; there was no value available for 6 seropositive patients.

TABLE 2
Characteristics of TB and control subjects

| | | |
|---|---|---|
| Train | Test | Total |

a. TB patient characteristics

| | | | | | | |
|---|---|---|---|---|---|---|
| Symptomatic (%): | 100 | (98) | 74 | (96.1) | 174 | (97.2) |
| Persistent Cough | 98 | (96) | 74 | (96.1) | 171 | (95.5) |
| Haemoptysis | 5 | (4.9) | 1 | (1.3) | 6 | (3.3) |
| Night sweats/fever | 68 | (66.6) | 53 | (66.8) | 121 | (67.6) |
| Weight loss (%) | | | | | | |
| ≧5% | 86 | (84.3) | 60 | (77.9) | 146 | (81.5) |
| <5% | 11 | (10.7) | 15 | (19.4) | 26 | (14.5) |
| Symptom duration pre-sampling | 122.6 | (13-449) | 129.5 | (12-754) | 126 | (12-754) |
| [mean(range)] | | | | | | |
| Smear Positive | 89 | (87.2) | 66 | (85.7) | 155 | (86.5) |
| Pulmonary disease | 77 | (75.4) | 64 | (83.1) | 141 | (78.7) |
| Extra-pulmonary disease | 2 | (1.9) | 2 | (2.6) | 4 | (2.2) |
| Pulmonary and extra-pulmonary | 22 | (21.5) | 11 | (14.2) | 33 | (18.4) |
| Abnormal CXR (%) | 95 | (93.1) | 67 | (87) | 162 | (90.5) |
| Cavitary Disease (%) | 66 | (64.7) | 49 | (63.6) | 115 | (64.2) |
| Previous BCG vaccination¹ (%) | 36 | (35.3) | 26 | (33.8) | 62 | (34.6) |
| Skin test positive² | 56 | (54.9) | 36 | (46.8) | 92 | (51.4) |

b. Control diagnostic groups³

| | | | | | | |
|---|---|---|---|---|---|---|
| Inflammatory bowel disease | 10 | (10.9) | 6 | (7.5) | 16 | (9.4) |
| Sarcoidosis | 6 | (6.5) | 7 | (8.8) | 13 | (7.6) |
| Respiratory infections | 27 | (29.6) | 24 | (30.3) | 51 | (30) |
| Other Infections: | | | | | | |
| Malaria (P. falciparum) | 4 | (4.4) | 3 | (3.8) | 7 | (4.1) |
| HAT (T. b. gambiense)⁴ | 10 | (10.9) | 9 | (11.3) | 19 | (11.1) |
| Others⁵ | 1 | (1.1) | 2 | (2.5) | 3 | (1.7) |
| Neurological disease⁶ | 13 | (14.2) | 13 | (16.4) | 26 | (15.2) |
| Autoimmune disease⁷ | 6 | (6.5) | 3 | (3.8) | 9 | (5.2) |
| Myeloma/monoclonal gammopathy | 2 | (2.2) | 3 | (3.8) | 5 | (2.9) |
| Healthy volunteers | 12 | (13.1) | 9 | (11.3) | 21 | (12.3) |

¹Definite history of BCG vaccination and/or presence of scar. Data missing from 38 patients.
²Mantoux reaction ≧15 mm greatest diameter of induration or Heaf grade ≧3. Data missing from 46 patients.
³12 control subjects were taking high dose systemic steroids (prednisolone ≧60 mg/day or dexamethasone ≧12 mg/day).
⁴9 patients with HAT had advanced (neurological disease) based on detection of parasites and/or >5 white cells/mm³ in CSF.
⁵visceral leishmaniasis (1), meningococcal septicaemia (1), staphylococcal cellulitis (1).
⁶cerebral neoplasia (12), cerebral abscess in association with infective endocarditis (1), myasthenia gravis (2), multiple sclerosis (5) and lumbar disc prolapse (6).
⁷rheumatoid arthritis (5), systemic lupus erythematosis (4), systemic sclerosis (1), overlap syndrome (1).

TABLE 3
Diagnostic Performance of classifiers

| | | Actual | | | | |
|---|---|---|---|---|---|---|
| Classifier | Output | TB | C | Accuracy % | Sensitivity % | Specificity % |
| Support Vector Machine | TB | 72 | 4 | 94.23 | 93.50 | 94.93 |
| Kernel: Gaussian | C | 5 | 75 | | | |
| Sigma = 0.00004 | | | | | | |
| Soft Margin = 10 [symbol] SVM_1 | | | | | | |
| ADTree + AdaBoost | TB | 72 | 7 | 92.30 | 93.50 | 91.13 |
| 100 iterations | C | 5 | 72 | | | |
| Weight threshold = 100 | | | | | | |
| [symbol] ADT_2 | | | | | | |
| C4.5Tree + AdaBoost | TB | 71 | 8 | 91.02 | 92.20 | 89.87 |
| 100 iterations | C | 6 | 71 | | | |
| Weight threshold = 100 | | | | | | |
| [symbol] C4.5_2 | | | | | | |
| Tree Classifier C5.0 | TB | 72 | 10 | 90.38 | 93.51 | 87.34 |
| Boost = 10, | C | 5 | 69 | | | |
| Global Pruning 25% | | | | | | |
| [symbol] C5.0_1 | | | | | | |
| Support Vector Machine | TB | 71 | 9 | 88.46 | 92.20 | 84.81 |
| Kernel: polynomial | C | 6 | 70 | | | |
| Dimension = 3 | | | | | | |
| Soft Margin = 1 [symbol] SVM_4 | | | | | | |
| SLP | TB | 68 | 12 | 86.54 | 88.31 | 84.81 |
| Normalized | C | 9 | 67 | | | |
| Shuffled Presentation | | | | | | |
| [symbol] SLP_3 | | | | | | |
| MLP [1 HL (111 N)] | TB | 65 | 9 | 86.53 | 84.41 | 88.60 |
| Learning rate = 0.3 | C | 12 | 70 | | | |
| Momentum = 0.2 | | | | | | |
| Normalized 500 epochs [symbol] MLP | | | | | | |

TB = tuberculosis;
C = controls.
ADTree = adaptive decision tree.
AdaBoost = adaptive boosting.
SLP = single layer perceptron.
MLP = multi layered perceptron.
HL = hidden layers.
N = neurons.
Key in italics and colors corresponds to name of classifier in FIG. 1a.

TABLE 4
Classifiers performance on selected mass cluster peaks and biomarkers

| | Features | Accuracy | Sensitivity | Specificity | TPR | FPR |
|---|---|---|---|---|---|---|
| Mass Peaks | 10 positive correlated and 10 negative correlated | 0.90 | 0.90 | 0.90 | 0.90 | 0.10 |
| | 199 (remaining) | 0.86 | 0.82 | 0.90 | 0.82 | 0.10 |
| | 10 positive correlated | 0.78 | 0.75 | 0.80 | 0.75 | 0.20 |
| | 209 (remaining) | 0.89 | 0.83 | 0.95 | 0.83 | 0.05 |
| | 10 negative correlated | 0.85 | 0.88 | 0.81 | 0.88 | 0.19 |
| | 209 (remaining) | 0.89 | 0.87 | 0.91 | 0.87 | 0.09 |
| Markers | Transthyretin | 0.73 | 0.85 | 0.61 | 0.85 | 0.39 |
| | CRP | 0.80 | 0.85 | 0.74 | 0.85 | 0.26 |
| | Neopterin | 0.73 | 0.78 | 0.67 | 0.78 | 0.33 |
| | SAA | 0.82 | 0.86 | 0.77 | 0.86 | 0.23 |
| | Neopterin - SAA | 0.74 | 0.77 | 0.71 | 0.77 | 0.29 |
| | CRP - SAA | 0.83 | 0.86 | 0.80 | 0.86 | 0.20 |
| | CRP - Neopterin | 0.80 | 0.78 | 0.83 | 0.78 | 0.17 |
| | Transthyretin - SAA | 0.81 | 0.92 | 0.70 | 0.92 | 0.30 |
| | Transthyretin - Neopterin | 0.80 | 0.95 | 0.65 | 0.95 | 0.35 |
| | Transthyretin - CRP | 0.82 | 0.92 | 0.71 | 0.92 | 0.29 |
| | Transthyretin - CRP - Neopterin | 0.84 | 0.82 | 0.86 | 0.82 | 0.14 |
| | Transthyretin - CRP - SAA | 0.82 | 0.92 | 0.72 | 0.92 | 0.28 |
| | Transthyretin - Neopterin - SAA | 0.80 | 0.92 | 0.67 | 0.92 | 0.33 |
| | CRP - Neopterin - SAA | 0.82 | 0.85 | 0.80 | 0.85 | 0.20 |
| | Transthyretin - CRP - Neopterin - SAA | 0.79 | 0.89 | 0.68 | 0.89 | 0.32 |

TABLE 5
Identification of Protein Markers

| | |
|---|---|
| SELDI- | |
| TOF/MS | |
| BIOMARKER | DATA DERIVED FROM 2D GELS |

| | | | |
|---|---|---|---|
| Mass | PE Mass | pI | ID from LC/MS/Ms |

Positive in TB

| | | | |
|---|---|---|---|
| 18394 | 18474 | 6.0 | Serum Albumin |
| | | | precurser |
| 11720 | 11718 | 6.5 | |
| 11454 | 11601 | 7.0 | Apo-A1 |
| | 11506 | 7.5 | |
| | 11698 | 8.8 | |

Negative in TB

| | | | |
|---|---|---|---|
| 13774 | 13851 | 5.7 | Transthyretin precurser |

TABLE 6
Protein Markers identified by 2D gel analysis

| | | |
|---|---|---|
| PE Mass | | |
| (accurate) | pI | ID from LC/MS/Ms |

Spots in TB gel

| | | |
|---|---|---|
| 8648 | 4.6 | APOA-2 precursor |
| 8771 | 4.6 | ApoA-2 |
| 16020 | 7.6 | Hemoglobin Beta |
| 13876 | 5.7 | Transthyretin precursor |
| | 4.25 | A2G1 (LRG1) |

Spots in Control gel

| | | |
|---|---|---|
| 13851 | 5.7 | Transthyretin precursor |
| | 9.3 | DEP Domain protein |
| | 6.5, 5.9 and 6.3 | Hypothetical protein |
| | | DFKZp667I032 |

Bold text denotes that the protein spot was more intense than the equivalent spot in the other gel.
Italic text denotes the protein spot was less intense than the equivalent spot in the other gel.

1. A method of diagnosing tuberculosis (TB) in a test subject, said method comprising:
(i) providing expression data of two or more markers in a subject, wherein at least two of said markers are selected from transthyretin, neopterin, C-reactive protein (CRP), serum amyloid A (SAA), serum albumin, apoliopoprotein-AI (Apo-AI), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) comparing said expression data to expression data of said marker from a group of control subjects, wherein said control subjects comprise patients suffering from inflammatory conditions other than TB, thereby determining whether or not said test subject has TB.

2. A method according to claim 1, wherein said group of control subjects is selected from two or more of patients with respiratory infections, patients with sarcoidosis, patients with inflammatory bowel disease, patients with malaria, patients with human African trypanosomiasis (HAT), patients with neurological disease, patients with autoimmune disease, patients with myeloma and healthy subjects.

3. A method of diagnosing tuberculosis (TB), said method comprising:
(i) providing expression data of two or more markers in a subject, wherein at least two of said markers are selected from transthyretin, neopterin, C-reactive protein (CRP), serum amyloid A (SM), serum albumin, apoliopoprotein-AI (Apo-AI), apolipoprotein-A2 (Apo-A2), hemoglobin beta, haptoglobin protein, DEP domain protein, leucine-rich alpha-2-glycoprotein (A2GL) and hypothetical protein DFKZp6671032; and
(ii) determining whether expression of said markers is indicative of TB.

4. A method according to claim 1, wherein one of said markers is transthyretin.

5. A method according to claim 4, wherein said markers comprise transthyretin, CRP and neopterin.

6. A method according to claim 1, wherein step (ii) is implemented using a computer system.

7. A method according to claim 6, wherein the computer system is programmed with a trained machine learning classifier.

8. A method according to claim 7, wherein said machine learning classifier is a support vector machine (SVM).

9. A method according to claim 3, wherein step (ii) comprises comparing expression of said markers in said subject to expression of said markers in a control subject.

10. A method according to claim 9, wherein the control subject is a patient suffering from an inflammatory condition other than TB.

11. A method according to claim 9, wherein said control subjects are selected from one or more of patients with respiratory infections, patients with sarcoidosis, patients with inflammatory bowel disease, patients with malaria, patients with human African trypanosomiasis (HAT), patients with neurological disease, patients with autoimmune disease, patients with myeloma and healthy subjects.

12. A method according to claim 1, wherein step (ii) comprises comparing expression of said markers in said subject to expression of said markers in a TB patient.

13. A method according to claim 12, wherein said TB patient has been diagnosed as having TB by culture of Mycobacterium tuberculosis.

14. A method according to claim 12, wherein one or more patient having TB and/or one or more control subject is HIV positive.

15. A method according to claim 1, wherein said markers comprise two or more of transthyretin, neopterin, CRP, SM, serum albumin and Apo-AI and one or more of apolipoprotein-A2, hemoglobin beta, haptoglobin protein, DEP domain protein, A2GL and hypothetical protein DFKZp6671032.

16. A method according to claim 1, wherein said expression data is obtained by capture of said markers on a surface and detection of the captured markers.

17. A method according to claim 16, wherein said surface is a surface enhanced laser desorption and ionization (SELDI) probe and said detection is by SELDI-time of flight mass spectroscopy (SELDI-TOF MS).

18. A method according to claim 17, wherein said markers comprise one or more positively correlated markers having m/z values of about M18394_{—}9, about M8952_{—}75, about M11720_{—}0, about M11454_{—}1, about M18591_{—}2, about M11488_{—}1, about M9076_{—}68, about M8895_{—}13 and about M10856_{—}8 and/or one or more negatively correlated markers having m/z values of about M4100_{—}03, about M3898_{—}52, about M13972_{—}1, about M3322_{—}01, about M2956_{—}45, about M5644_{—}96, about M3939_{—}63, about M4056_{—}39 and about M6649_{—}74.

19. A method according to claim 18, wherein said markers comprise all said positively correlated markers and/or all said negatively correlated markers.

20. A method according to claim 16, wherein said surface comprises specific binding reagents for said markers and said detection is by immunoassay.

21. A computer-implemented method of diagnosing TB, said method comprising:
(i) inputting expression data of two or more markers in a subject; and
(ii) determining whether expression of said markers is indicative of TB using a computer system programmed with a trained support vector machine (SVM) thereby diagnosing whether or not said patient has TB.

22. A method according to claim 21, wherein said SVM has been trained using data obtained from patients diagnosed as having TB by culture of Mycobacterium tuberculosis and from control subjects selected from one or more of patients with respiratory infections, patients with sarcoidosis, patients with inflammatory bowel disease, patients with malaria, patients with human African trypanosomiasis (HAT), patients with neurological disease, patients with autoimmune disease, patients with myeloma and healthy subjects.

23. A method of training a support vector machine (SVM) classifier to diagnose tuberculosis (TB), said method comprising:
(i) providing training data which comprises:
(a) training data relating to two or more markers in each of a first set of TB patients; and
(b) training data relating to said two or more markers in each of a first set of control subjects;
(ii) using a SVM to discriminate the training data of TB patients from the training data of control subjects;
thereby training the SVM to diagnose TB.

24. A method according to claim 23, said method further comprising:
(iii) providing testing data which comprises:
(a) testing data relating to said two or more markers in each of a second set of TB patients; and
(b) testing data relating to said two or more markers in each of a second set of control subjects;
(iv) determining the ability of the SVM to correctly discriminate the testing data of TB patients from the testing data of control subjects.

25. A method according to claim 23, wherein said control subjects are selected from one or more of patients with respiratory infections, patients with sarcoidosis, patients with inflammatory bowel disease, patients with malaria, patients with human African trypanosomiasis (HAT), patients with neurological disease, patients with autoimmune disease, patients with myeloma and healthy subjects.

26. A method according to claim 23, wherein said training data and said testing data are obtained by SELDI analysis.

27. A method according to claim 23, wherein said training and said testing data are obtained by immunoassay analysis.

28. A method according to claim 23, wherein at least one of said markers is selected from CRP, neopterin, SAA, transthyretin, serum albumin and Apo-AI.

29. A method according to claim 28, wherein said markers comprise CRP, transthyretin and neopterin.

30. A method according to claim 23, wherein at least one of said markers is selected from Apo-A2, hemoglobin beta, haptoglobin protein, DEP domain protein, A2GL and hypothetical protein DFKZp6671032.

31. An apparatus arranged to perform a method according to claim 21 comprising:
(i) means for receiving expression data of two or more markers in a sample from a subject;
(ii) a module for determining whether said data is indicative of TB, wherein said module comprises a trained machine learning classifier capable of distinguishing data from a TB patient from data from a control subject; and
(iii) means for indicating the results of said determination.

32. An apparatus according to claim 31, which is a personal computer.

33. A computer program executable by a computer system, the computer program being capable, on execution by the computer system, of causing the computer system to perform a method claim 21.

34. A storage medium storing in a form readable by a computer system having a computer program according to claim 33.

35. A kit for diagnosing TB comprising:
(i) means for detecting two or more markers; and
(ii) a storage medium according to claim 34.

36. A kit for diagnosing TB comprising:
(i) means for detecting two or more markers;
(ii) instructions for inputting data relating to detection of said markers into an apparatus according to claim 31.

37. A kit according to claim 35, wherein said markers are selected from transthyretin, neopterin, CRP, SAA, serum albumin, Apo-AI, Apo-A2, hemoglobin beta, haptoglobin protein, DEP domain protein, A2GL and hypothetical protein DFKZp6671032.

38. A kit for diagnosing TB comprising:
(i) means for detecting two or more markers selected from transthyretin, neopterin, C-reactive protein (CRP), serum amyloid A (SAA), serum albumin, apoliopoprotein-AI (Apo-AI), apolipoprotein-A2 (Apo-A2), hemoglobin beta, haptoglobin protein, DEP domain protein, leucine-rich alpha-2-glycoprotein (A2GL) and hypothetical protein DFKZp6671032.

39. A kit according to claim 35, wherein said means of detecting two or more markers comprises a capture surface.

40. A kit according to claim 39, wherein said capture surface is a protein chip.

41. A kit according to claim 39, wherein said capture surface comprises specific binding reagents for said markers.

42. A kit according to claim 41, wherein said specific binding reagents are antibodies or antibody fragments.

43. A kit according to claim 37, wherein said markers are transthyretin, neopterin and CRP.

44. A method according to claim 1 further comprising administering to a patient diagnosed as having TB, a medicament for treatment of TB.

45. A method of identifying an agent for the treatment of TB, said method comprising:
(i) contacting a test agent with transthyretin, neopterin, CRP, SAA, serum albumin, Apo-AI, Apo-A2, hemoglobin beta, haptoglobin, DEP domain protein or A2GL; and
(ii) determining whether test agent modulates the activity of said transthyretin, neopterin, CRP, SAA, serum albumin, Apo-AI, Apo-A2, hemoglobin beta, haptoglobin, DEP domain protein or A2GL thereby determining whether or not said test agent is suitable for use in the treatment of TB.

46. A method of identifying an agent for the treatment of TB, said method comprising:
(i) contacting cells ex vivo or in vivo with Mycobacterium tuberculosis and a test agent;
(ii) monitoring expression of one or more TB markers selected from transthyretin, neopterin, CRP, SM, serum albumin, Apo-AI, Apo-A2, hemoglobin beta, haptoglobin, DEP domain protein and A2GL; and
(iii) determining whether test agent modulates the expression of said one or more test markers,
thereby determining whether or not said test agent is suitable for use in the treatment of TB.
